(19) United States
(12) Patent Application Publication
(10) Pub. No.: US 2011/0052565 A1
(43) Pub. Date: Mar. 3, 2011

(54) NON-AGGREGATING HUMAN VH DOMAINS

(75) Inventors: Mehdi Arbabi-Ghahroudi, Ottawa (CA); Jamshid Tanha, Ottawa (CA)

(73) Assignee: NATIONAL RESEARCH COUNCIL OF CANADA, Ottawa, ON (CA)

(21) Appl. No.: 12/808,090

(22) PCT Filed: Dec. 22, 2008
(86) PCT No.: PCT/CA08/02273

§ 371 Date: Sep. 15, 2010

Related U.S. Application Data
(60) Provisional application No. 61/016,139, filed on Dec. 21, 2007.

(51) Int. Cl. A61K 39/395; C07K 16/00; C40B 40/10; C40B 30/04; C07H 21/04; C12N 15/63; G01N 33/53; A61P 31/00

(52) U.S. Cl. 424/130.1; 530/387.1; 506/18; 506/9; 536/23.53; 435/320.1; 435/7.92

(57) ABSTRACT

The present invention relates to non-aggregating VH domains or libraries thereof. The V_{H} domains comprise at least one disulfide linkage-forming cysteine in at least one complementarity-determining region (CDR) and an acidic isoelectric point (pI). A method of increasing the power or efficiency of selection of non-aggregating V_{H} domains comprises panning a phagemid-based V_{H} domain phage-display library in combination with a step of selecting non-aggregating phage-V_{H} domains. Compositions of matter comprising the non-aggregating V_{H} domains, as well as methods of use are also provided.

### FIELD OF THE INVENTION

The present invention relates to antibody heavy chain variable domains. In particular, the invention relates to non-aggregating human V_{H} domains and methods of preparing and using same.

### BACKGROUND OF THE INVENTION

Antibodies play an important role in diagnostic and clinical applications for identifying and neutralizing pathogens. An antibody is constructed from paired heavy and light polypeptide chains. When an antibody is correctly folded, each chain folds into a number of distinct globular domains joined by more linear polypeptide sequences. For example, the light chain folds into a variable (V_{L}) and a constant (C_{L}) domain. Interaction of the heavy and light chain variable domains (V_{H} and V_{L}) results in the formation of an antigen binding region (Fv). Generally, both V_{H} and V_{L} are required for optimal antigen binding, although heavy chain dimers and amino-terminal fragments have been shown to retain activity in the absence of light chain.

The light and heavy chain variable regions are responsible for binding the target antigen and can therefore show significant sequence diversity between antibodies. The constant regions show less sequence diversity, and are responsible for binding a number of natural proteins to elicit important biochemical events. The variable region of an antibody contains the antigen binding determinants of the molecule, and thus determines the specificity of an antibody for its target antigen. The majority of sequence variability occurs in the complementarity-determining regions (CDRs). There are six CDRs total, three each per variable heavy and light chain, designated V_{H} CDR1, V_{H} CDR2, V_{H} CDR3, V_{L} CDR1, V_{L} CDR2, and V_{L} CDR3. The region outside of the CDRs is referred to as the framework region (FR). This characteristic structure of antibodies provides a stable scaffold upon which substantial antigen-binding diversity can be explored by the immune system to obtain specificity for a broad array of antigens.

The immune repertoire of camelids (camels, dromedaries and llamas) is unique in that it possesses unusual types of antibodies referred to as heavy-chain antibodies (Hamers et al, 1993). These antibodies lack light chains and thus their combining sites consist of one domain, termed V_{H}H. Single domain antibodies (sdAbs) have also been observed in shark and are termed VNARs.

sdAbs provide several advantages over single-chain Fv (scFv) fragments derived from conventional four-chain antibodies. Single domain antibodies are comparable to their scFv counterparts in terms of affinity, but outperform scFvs in terms of solubility, stability, resistance to aggregation, refoldability, expression yield, and ease of DNA manipulation, library construction and 3-D structural determinations. Many of the aforementioned properties of sdAbs are desired in applications involving antibodies. However, the non-human nature of naturally-occurring sdAbs (camelid V_{H}Hs and shark VNARs) limits their use in humans due to immunogenicity. In this respect, human V_{H} domains (“V_{H}s”) are ideal candidates for immunotherapy in humans. While naturally-occurring single domain antibodies can be isolated from libraries (for example, phage display libraries) by panning based solely upon binding property as the selection criterion (Arbabi-Ghahroudi et al., 1997; Lauwereys et al., 1998), this is not true in the case of human V_{H}s, as they are prone to forming high molecular weight aggregates in solution.

Attempts have been made to isolate non-aggregating V_{H}s (Davies et al., 1994; Tanha et al., 2001; Tanha et al., 2006; Jespers et al., 2004a; To et al., 2005). One prior art method involves phage display libraries and sequential steps of subjecting the library to heat to denature phage-displayed V_{H}s; to cooling; and to target antigens in the binding stage of the panning (Jespers et al., 2004a). V_{H}s with reversible unfolding characteristic regain their binding during the cooling step and are subsequently selected during the binding step, however the ones with irreversible denaturation characteristic, which include insoluble V_{H}s, are lost to aggregation and are eliminated. The method is conducted with phage vector-based phage display libraries. However, this approach requires multivalent display of V_{H}s on the phage surface; it has been demonstrated that this method was effective with phage vector-based display libraries, but not in a monovalent display format bestowed with phagemid vector-based systems (for phage display systems and their characteristics, see Winter et al., 1994; Bradbury and Marks, 2004).

It is desirable to isolate V_{H}s that are antigen-specific, soluble and structurally stable for use in clinical and diagnostic applications. Thus, there is a need in the art for non-aggregating human V_{H} domains and methods of producing non-aggregating human V_{H} domains that mitigate the disadvantages of the prior art.

### SUMMARY OF THE INVENTION

In one aspect, the present invention comprises antibody heavy chain variable (V_{H}) domains. In view of the problems associated with known V_{H} domains and methods of isolating same, novel human V_{H} domains have been engineered that display beneficial properties for clinical and diagnostic applications.

Accordingly, in one aspect, the present invention comprises a non-aggregating human V_{H} domain or libraries thereof comprising at least one disulfide linkage-forming cysteine in at least one complementarity determining region, and having an acidic isoelectric point.

The V_{H} domain may be soluble, capable of reversible thermal unfolding, or capable of binding to protein A. The V_{H} domain may have at least one cysteine in CDR1, and/or it may have at least three cysteines in CDR3. The V_{H} may form non-canonical disulfide linkages within one CDR, e.g., intra-CDR, or between CDRs, e.g., inter-CDR. These intra- or inter-CDR disulfide linkages may form extended loops. The V_{H} may be an enzyme inhibitor, and the inhibition may be through the extended loops (or CDR) formed by the disulfide linkages.

In a further embodiment, the V_{H}s of the present invention may be characterized by the presence of an acidic residue (aspartate or glutamate) at position 32 in CDR1. The VH domain may also have an acidic isoelectric point of below 6.

In another aspect of the present invention, the non-aggregating V_{H} domain or libraries thereof comprise human framework sequences and at least one CDR from a different species; for example, the V_{H} domain may comprise human framework sequences, and camelid CDR sequences. Alternatively, and in a further non-limiting example, the V_{H} domain may comprise human framework sequences, human CDR1/HI, human CDR2/H2, and camelid CDR3/H3.

The non-aggregating V_{H} domain or libraries thereof may also comprise mixed randomized sequences or libraries.

In another embodiment, the non-aggregating V_{H} domain or libraries thereof comprise a sequence selected from any one of SEQ ID NOs: 24-90, SEQ ID NOs:101-131, SEQ ID NOs: 132-162, and combinations thereof.

In a further aspect, the invention may comprise non-aggregating V_{H} domain or libraries thereof may be based on human V_{H} germline sequences, for example 1-f V_{H} segment, 1-24 V_{H} segment and 3-43 V_{H} segment. Alternatively, the V_{H}s and the libraries thereof of the present invention may be based on camelid V_{H} cDNAs or camelid germline V_{H} segments with acidic pIs. In another alternative, the V_{H}s and the libraries thereof may be based on camelid V_{H}H cDNAs or camelid germline V_{H}H segments with acidic pIs.

In another embodiment, the V_{H} domain comprises one of huVHAm302 (SEQ ID NO:15), huVHAm309 (SEQ ID NO:17), huVHAm316 (SEQ ID NO:19), huVHAm303 (SEQ ID NO:164), huVHAm304 (SEQ ID NO:16), huVHAm305 (SEQ ID NO:15165 huVHAm307 (SEQ ID NO:166), huVHAm311 (SEQ ID NO:167), huVHAm315 (SEQ ID NO:18), huVHAm301 (SEQ ID NO:163), huVHAm312 (SEQ ID NO:168), huVHAm320 (SEQ ID NO:171), huVHAm317 (SEQ ID NO:170), huVHAm313 (SEQ ID NO:169), huVHAm431 (SEQ ID NO:23), huVHAm427 (SEQ ID NO:21), huVHAm416 (SEQ ID NO:20), huVHAm424 (SEQ ID NO:175), huVHAm428 (SEQ ID NO:22), huVHAm430 (SEQ ID NO:176), huVHAm406 (SEQ ID NO:172), huVHAm412 (SEQ ID NO:173) or huVHAm420 (SEQ ID NO:174).

In one embodiment, the V_{H} domain is isolated from a phagemid-based phage display library. The isolation of the V_{H} domain may include a selection step that either enhances the power or efficiency of selection for non-aggregating V_{H} domains.

In a specific embodiment, the present invention provides a V_{H} domain or library thereof, wherein a) the V_{H} domain is based on HVHP430 (SEQ ID NO:1); b) the Cys at positions 99 and 100d of CDR3 are maintained; c) the remaining 14 amino acid residues of CDR3 are randomized; d) amino acid residue 94 is randomized; and e) the 8 amino acid residues of CDR1/H1 are randomized.

In another aspect, the invention comprises a V_{H} domain library, wherein a) the V_{H} domain is based on HVHP430 (SEQ ID NO:1); b) the amino acid residues at 93-102 (93/94-CDR3) positions are derived from llama V_{H}Hs; c) the 8 amino acid residues of CDR1/H1 are randomized.

In yet another embodiment, the present invention encompasses a V_{H} domain or library thereof, wherein a) the V_{H} domain is based on HVHP430 (SEQ ID NO:1); b) the CDR3 comprises a sequence selected from SEQ ID NOs:24-90 and SEQ ID NOs:33-63; c) the 8 amino acid residues of CDR1/H1 are randomized.

In another aspect, the present invention also provides a method of increasing the power or efficiency of selection of non-aggregating V_{H} domains by:
a) providing a phagemid-based V_{H} domain phage-display library, wherein the library is produced by multivalent display of V_{H} domains on the surface of phage; and
b) panning, using the phage-V_{H} domain library and a binding target,
where the method comprises a step of selecting non-aggregating phage-V_{H} domains. The selection step may be a step of subjecting the phage-V_{H} domain library to a heat denaturation/re-naturation, which would occur prior to the step of panning (step b)). Alternatively, the selection step may be a step of sequencing individual clones to identify the V_{H} with acidic pIs occurring following panning (step b)). In yet another alternative, the selection step may comprise both heat denaturation/re-naturation and sequencing of individual clones to identify the V_{H} with acidic pIs.

In one embodiment, the method may further comprise a step of isolating specific V_{H} domains from the phagemid-based V_{H} domain phage-display library.

In an alternative embodiment, the method may comprise the steps of:
c) providing a phage vector-based V_{H} domain phage-display library, wherein the library is produced based on a V_{H} domain scaffold having an acidic pI;
d) panning, using the phage-V_{H} domain library and a target; and
e) sequencing individual clones to identify V_{H} domains having an acidic pI.

The V_{H} domain scaffolds for the described method may be based on human germline sequences with acidic pI, camelid V_{H} cDNAs, camelid germline V_{H} segments with acidic pIs, camelid V_{H}H cDNAs, or camelid germline V_{H}H segments with acidic pIs. Specific V_{H} domains from the phage vector-based V_{H} domain phage-display library may be isolated. The method as described above may further comprise a step of isolating specific V_{H} domains from the phage vector-based V_{H} domain phage-display library.

In another aspect, the present invention comprises nucleic acids encoding the V_{H}s of the present invention, vector comprising the nucleic acid, and a host cell comprising the nucleic acid or the vector. In another aspect, V_{H}s may be expressed in a host including, but not limited to any yeast strains.

In yet another aspect, the invention comprises a pharmaceutical composition comprising one or more V_{H} domains in an effective amount for binding thereof to an antigen, and a pharmaceutically-acceptable excipient.

In another aspect, the invention comprises a use of a V_{H} domain in the preparation of a medicament for treating or preventing a medical condition by binding to an antigen.

The invention also provides a method of treating a patient, comprising administering a pharmaceutical composition comprising one or more V_{H} domains to a patient in need of treatment.

In still another aspect, the invention provides a kit comprising one or more V_{H} domains and one or more reagents for detection and determination of binding of the one or more V_{H} domains to a particular antigen in a biological sample.

The V_{H}s of the present invention may also be used in a high-throughput screening assay, such as microarray technology, in which the use of the V_{H} domain is advantageous to conventional IgG due to its size and stability.

Embodiments of the present invention utilize a heat denaturation panning approach to a phagemid-based V_{H} phage display library. Phagemid vector-based phage display systems offer many advantages over phage vector-based systems, including ease-of-use, suitability for isolation of high affinity binders, and rapid antibody expression and analysis. In addition, the use of helper phages result in multivalent display (Rondot et al., 2001; Baek, et al., 2002; Soltes et al.,2003), and therefore in a high yield of binders, fewer rounds of panning and more efficient enrichment. Moreover, with a phagemid vector system, switching between monovalent and multivalent formats can be accomplished at will, by using the appropriate type of helper phage (Rondot et al., 2001; O'Connell et al., 2002; Kirsch et al., 2005).

V_{H}s of the present invention are characterized by non-aggregation and reversible thermal unfolding properties. The methods of the present invention combines selection for the biophysical properties mentioned above offered by phage vector-based display libraries (Jespers, et al., 2004) and the convenience of constructing large-size libraries with phagemid vectors, resulting in a more efficient selection for non-aggregating binders by tapping into larger sequence space. The present approach can also be used to simultaneously select for (i) non-aggregation and (ii) high affinity by alternating between panning in a multivalent display format with heat denaturation and in a monovalent display format. The presently described selection method can be applied to phagemid libraries with the aforementioned attribute to improve the enrichment not only for non-aggregating binders, but also for those with reversible thermal unfolding properties.

The present invention shows successful extension of the heat denaturation approach (Jespers et al., 2004) to selection of non-aggregating V_{H}s from a large synthetic human V_{H} library in a phagemid vector format. When panned in a multivalent display format, through phage rescue with hyperphage (M13KO7ΔpIII helper phage), and with a heat denaturation step, the library yielded non-aggregating V_{H}s that demonstrated reversible thermal unfolding. Selection was characterized by enrichment for V_{H}s with acidic pIs and/or inter-CDR1-CDR3 disulfide linkages. The library design included a feature to increase the frequency of enzyme-inhibiting V_{H}s in the library.

Additional aspects and advantages of the present invention will be apparent in view of the following description. The detailed description and examples, while indicating preferred embodiments of the invention, are given by way of illustration only, as various changes and modifications within the scope of the invention will become apparent to those skilled in the art in light of the teachings of this invention.

### BRIEF DESCRIPTION OF THE DRAWINGS

These and other features of the invention will now be described by way of example, with reference to the appended drawings, wherein:

FIG. 1 illustrates (i) molecular mass profiles obtained by mass spectrometry of unreduced/alkylated (unred/alk) and reduced/alkylated (red/alk) HVHP430 V_{H} and (ii) the results of alkylation reaction/mass spectrometry experiments for HVHP430 and four anti-α-amylase V_{H}s. The theoretical values for the number of disulfide linkages are calculated based on the assumption that all the CDR Cys residues would be involved in disulfide linkage formation. The “Total” number of disulfide linkages is the sum of the intra-/inter-CDR disulfide linkages and the canonical disulfide linkage between Cys 22 and Cys 92.

FIG. 2A shows the amino acid sequence of HVHP430 (SEQ ID NO:1), with the randomized residues underlined. H1 (hypervariable loop 1) spans residues 26-32 (GFTFSNY; SEQ ID NO:177) (Chothia, et al., 1992). CDR1 (complementarity-determining region 1) overlaps with H1 and spans residues 31-35 (NYAMS; SEQ ID NO:178). CDR and framework region (FR) designations and numbering are according to Kabat et al (1991).

FIG. 2B shows schematic steps in the construction of the human V_{H} phage display library.

FIG. 3 shows a map of pMED1 phagemid vector, with the nucleotide sequence of the multiple cloning site and its immediate surroundings shown in (ii). RBS, ribosome binding site; L, left; R, right; HA, heaemagglutinin; fd, filamentous bacteriophage, fd.

FIG. 4 shows size exclusion chromatograms of the V_{H}s isolated by panning the V_{H} library against α-amylase in a monovalent display format (A) or a multivalent display format with a heat denaturation step (B). (A) huVHAm455 (dotted line) precipitated highly and thus gave low absorbance signals. (B) huVHAm304: dotted-dashed line; huVHAm309: dotted line; huVHAm428: solid line; huVHAm416: dashed line. (C) Expansion of FIG. 4B to show an improved resolution of the peaks.

FIG. 5 shows graphs illustrating the aggregation tendencies of V_{H}s in terms of the percentage of their monomeric contents.

FIG. 6 shows steps in the determination of the identity of the amino acid coded by the amber codon at position 32 of huVHAm302. (A) Sequence of huVHAm302 as determined by mass spectrometry. Spaces define the boundaries between FRs and CDRs (see FIG. 2A). The determined peptide sequences from the analysis of the tryptic digest of huVHAm302 using nanoRPLC-MS/MS are boldfaced (see also FIG. 6B). The amber codon at position 32 was found to code for an E (underlined). The N-terminus of huVHAm302 was determined as pyroglutamine (pyroQ). The N-terminal tryptic peptide sequence, pyroQVQLVESGGGLIKPGGSLR (SEQ ID NO:179), was obtained from the MS/MS spectrum of a prominent doubly protonated ion at m/z 939.50 (2+) (data not shown). Moreover, the N-terminal fragment ions from the CID of the protonated protein ion at m/z 1413.71(11+) showed the N-terminus of huVHAm302 as pyroQ as well (data not shown). The determined molecular weight of the protein (15,541.2 Da) also indicated that the N-terminus of the protein is pyroglutamine. The C-terminal tryptic peptide ion at m/z 585.91 (3+) from LSEEDLNHHHHHH (SEQ ID NO: 180) was prominent in the survey scan of the DDA experiment. Peptides having amino acids attached after the C-terminal histidine were not observed. In addition, collision induced dissociation (CID) of the protein ion [M+11H] 11+ at m/z 1413.71 (11+) was performed and the C-terminal tryptic peptide sequence VTVSSGSEQKLSEEDLNHHHHHH (SEQ ID NO:181) was obtained from the C-terminal fragment ions of the protein (MS/MS data not shown). (B) MS/MS spectrum of the doubly protonated ion at m/z 1036.47 (2+) for the tryptic peptide LSCamAASGDTVSDESMTWVR (SEQ ID NO:13; residues 20-38 of huVHAm302). The amber-coded amino acid, E, at position 32 is underlined. The mass spectrometry experiments also showed that the CDR3 Cys residues formed a disulfide linkage.

FIG. 7 shows SDS-PAGE analysis of V_{H}s (huVHAm431, huVHAm416) isolated by panning the V_{H} library against α-amylase by the heat denaturation method (arrow denotes the disulfide-mediated dimeric V_{H}. R: reduced; NR: not reduced).

FIG. 8 shows sensorgram overlays showing the binding of native (thick lines) and refolded (thin lines) huVHAm309 (A) and huVHAm416 (B) to immobilized protein A at 0.1, 0.2, 0.3, 0.4, 0.5, 1 and 2 μM (huVHAm309) and 0.1, 0.2, 0.3, 0.4, 0.5, 1, 2 and 4 μM (huVHAm416).

FIG. 9 shows binding analyses by ELISA of V_{H}s identified by the heat denaturation panning approach against α-amylase, with (A) binding of V_{H}s against immobilized α-amylase (dotted columns) and bovine serum albumin, BSA (checkered columns) and (B) binding of horseradish peroxidase-protein A conjugate to immobilized V_{H}s and BSA control. In both A and B, binding to BSA is at a background level.

FIG. 10 shows aspects of determining enzyme inhibition activity of anti-α-amylase V_{H}s. (A) α-amylase activity, measured as Δ405 nm, as a function of time. A clear inhibition can be seen with the amylase binder huVHAm302 (filled square) and not with the control V_{H} HVHP430 (Filled triangle). (B) Residual activity of α-amylase in the presence of various concentrations of anti-α-amylase V_{H}s. Only huVHAm302 acts as an enzyme inhibitor at all the V_{H} concentrations tested. Filled square: huVHAm302; open square: huVHAm428; filled circle: huVHAm304; open circle: huVHAm416.

FIGS. 11A-F are graphs illustrating theoretical pI distribution for L. glama cDNA V_{H}Hs of subfamilies V_{H}H1, V_{H}H2 and V_{H}H3, C. dromedarius cDNA V_{H}Hs, germline V_{H}H segments and germline V_{H} segments, human germline V_{H} segments and the HVHP430 library V_{H}s.

FIG. 12A shows a sample of CDR3 sequences from the llama V_{H}H CDR3 plasmid library with the CDR3 sequences derived from V_{H}H2 subfamily marked by asterisks; cysteine residues are underlined. The numbering system is that described by Kabat et al. (1991). FIG. 12B shows the length distribution of a sample of CDR3 sequences from the llama V_{H}H CDR3 plasmid library; the horizontal line denotes both the mean CDR3 length as well as the median (M).

FIG. 13 shows a CDR3 length distribution of a sample of V_{H}s from HVHP430LGH3 V_{H} phage display library, from which thirty-one V_{H}s were analyzed; the horizontal line denotes both the mean CDR3 length as well as the median (M).

FIG. 14 shows sequences for acidic human germline V_{H} segments.

### DETAILED DESCRIPTION OF THE INVENTION

The present invention relates to antibody heavy chain variable domains. In particular, the invention relates to non-aggregating human V_{H} domains and methods of isolating same.

The present invention comprises non-aggregating human V_{H} domains and libraries thereof, having at least one disulfide linkage-forming cysteine in at least one complementarity-determining region and having an acidic isoelectric point. The V_{H} domain as just described may also be soluble, capable of reversible thermal unfolding, and/or capable of binding to protein A. The V_{H} domain may comprise at least one cysteine in CDR1. The V_{H} domain as described may comprise at least three cysteines in CDR3.

The V_{H}s may display high solubility and/or reversible thermal unfolding. They may also be capable of binding to protein A. In a specific, non-limiting embodiment, the human V_{H} domain has an isoelectric point of below 6. The V_{H} domains and libraries thereof of the present invention may further comprise an Asp or Glu at position 32 of H1/CDR1 or other positions in H1/CDR1 or in H1/CDR1, H2/CDR2 or H3/CDR3.

As used herein, “V_{H} domain” or “V_{H}” refers to an antibody heavy chain variable domain. The term includes naturally-occurring V_{H} domains and V_{H} domains that have been altered through selection or engineering to change their characteristics including, for example, stability or solubility. The term includes homologues, derivatives, or fragments that are capable of functioning as a V_{H} domain.

As is known to one of skill in the art, a V_{H} domain comprises three “complementarity determining regions” or “CDRs”; generally, each CDR is a region within the variable heavy chain that combines with the other CDR to form the antigen-binding site. It is well-known in the art that the CDRs contribute to binding and recognition of an antigenic determinant. However, not all CDRs may be required for binding the antigen. For example, but without wishing to be limiting, one, two, or three of the CDRs may contribute to binding and recognition of the antigen by the V_{H} domains of the present invention. The CDRs of the V_{H} domain are referred to herein as CDR1, CDR2, and CDR3.

The numbering of the amino acids in the V_{H} domains of the present invention is done according to the Kabat numbering system, which refers to the numbering system used for heavy chain variable domains or light chain variable domains from the compilation of antibodies in Kabat et al., Sequences of Proteins of Immunological Interest, 5th Ed. Public Health Service, National Institutes of Health, Bethesda, Md. (1991). This system is well-known to one of skill in the art, and may be determined for a given antibody by alignment at regions of homology of the sequence of the antibody with a “standard” Kabat numbered sequence. The positions of the CDRs in V_{H}s, according to Kabat numbering are as follows: CDR1—residues 31-35B; CDR2—residues 50-65; and CDR3—residues 95-102.

V_{H} domains are also characterized by hypervariable regions, labelled H1, H2 and H3, which overlap the CDRs. H1 is defined as residues 26-32, H2 is defined as 52-56, and H3 is defined as residues 95-102 (http://www.bioinf.org.uk/abs/). The hypervariable regions are directly involved in antigen binding.

The V_{H} domains and libraries thereof of the present invention comprise at least one disulfide linkage-forming cysteine in at least one CDR. By the term “disulfide linkage-forming cysteine” it is meant a cysteine that forms a disulfide bridge (also referred to as “disulfide bond” or “disulfide linkage”) with another cysteine through oxidation of their thiol groups. Without wishing to be bound by theory, disulfide bridges help proteins and enzymes maintain their structural configuration. In particular, V_{H} domains comprise a canonical (i.e., highly conserved) disulfide bond between Cys 22 and Cys 92. In addition to this canonical disulfide bond, the V_{H}s of the present invention comprise at least one non-canonical disulfide bond. The latter may be at any non-canonical position in the V_{H} structure; for example, the non-canonical disulfide bond may be in the framework region, in a CDR, in the hypervariable loop, or any combination thereof.

In one embodiment, there is an even number of disulfide linkage-forming Cys. For example, and not wishing to be limiting in any manner, there may be at least one disulfide linkage-forming Cys in CDR1; in another non-limiting example, there may be at least one Cys in CDR3; in yet another non-limiting example, there may be at least three Cys in CDR3. The disulfide linkage-forming Cys of the V_{H} domains may form intra-CDR disulfide bonds or inter-CDR disulfide bonds. For examples, and without wishing to be limiting, the Cys residues in CDR3 of V_{H}s form intra-CDR disulfide linkages; in another non-limiting example, the Cys residues in CDR1 and CDR3 of V_{H}s form inter-CDR disulfide linkages.

Furthermore, and without wishing to be bound by theory, the non-canonical disulfide linkages in the CDR of the V_{H} of the present invention may be useful in producing enzyme inhibitors; specifically, the disulfide linkage(s) may form protruding CDR loops, and particularly CDR3 loops, for accessing cryptic epitopes or enzyme active sites. Non-canonical disulfide linkages have also been shown to be important in single domain antibody stability (Nguyen et al., 2000; Harmsen et al., 2000; Muyldermans et al., 1994; Vu et al., 1997; Diaz et al., 2002), as well as in shaping the combining site for novel topologies and increased repertoire diversity.

The generation of antibody-based inhibitors to enzymes and proteases that are involved in the pathobiology of a number of disease states is of particular interest from a pharmaceutical standpoint. Human V_{H} domains are superior for therapeutic applications due to their expected lower immunogenicity, small size, and stability.

However, human V_{H} domains tend to form high molecular weight aggregates in solution. These include structures that are not soluble as monomers and show non-specific interactions to other molecules or surfaces, sometimes refers to as “stickiness”. A V_{H} domain can form dimeric, or multimeric or high molecular weight aggregates, none of these are desirable or useful. The term “non-aggregating” refers to the reduced tendency or inability of the V_{H} domain to form such aggregates. The V_{H} domains of the present invention are non-aggregating. This is verified by elution on a gel filtration column, for example but no limited to Superdex™ 75 column, where the V_{H} domain is essentially monomeric. By “essentially monomeric”, it is mean that 95%, 96%, 97%, 98%, 99%, or 100% of the V_{H} domains elute as monomers. Preferably, the non-aggregating V_{H} domains of the present invention are stable and do not precipitate over time.

The V_{H} domains and libraries thereof of the present invention also have acidic pI. The term “pI” or “isoelectric point” means the pH at which the V_{H} domain carries no net electrical charge. Generally, solubility is at a minimum when the pH is at the pI. An acidic isoelectric point may be below 7; for example, the acidic pI may be below 7, 6, 5, 4, 3, 2, or 1, or any value therebetween, or within a range described by these values; in a non-limiting example, the pI of the V_{H} domains of the present invention is below 6. A neutral pI is 7, and a basic pI is above 7. Without wishing to be limiting, the acidic pI of the V_{H} domains of the present invention originates primarily from non-randomized regions, including, for example, the framework regions.

The “solubility” of the V_{H} of the present invention refers to its ability to dissolve in a solvent, as measured in terms of the maximum amount of solute dissolved in a solvent at equilibrium. The V_{H} of the present invention is soluble in monomeric form, with no stickiness. The V_{H} domains as presently described are soluble in an aqueous buffer, for example, but not limited to Tris buffers, PBS buffers, HEPES buffers, carbonate buffers, or water.

The V_{H} of the present invention may also exhibit “reversible thermal unfolding”. Thermal unfolding refers to the temperature-induced unfolding of a molecule from its native, folded conformation to a secondary, unfolded conformation. Thermal unfolding is reversible if the molecule can be restored from the secondary, unfolded conformation to its native, folded conformation. Reversible thermal unfolding is measured by the thermal refolding efficiency (TRE) of a molecule. The non-aggregating V_{H} domains as described above may show higher TRE than aggregating V_{H} domains and refold to their native state more efficiently. The temperature at which the present V_{H}s unfold will vary depending on the nature of the V_{H} and on its melting temperature. In general, most V_{H} will be unfolded at temperatures above 60° C., above 85° C., or above 90° C. In a non-limiting example, the V_{H} s of the present invention may be able to regain antigen specificity following prolonged incubations at temperatures above 80° C., or even above 90° C.

The V_{H}s may also bind to protein A, a molecule well-known to those of skill in the art. Protein A is often coupled to other molecules without affecting the antibody binding site; for example, and without wishing to be limiting, protein A may be coupled to fluorescent dyes, enzymes, biotin, colloidal gold, radioactive iodine, and magnetic, latex, and agarose beads. Protein A can also be immobilized onto a solid support and used as a reliable method for purifying immunoglobulin from mixtures—for example from serum, ascites fluid, or bacterial extract—or coupled with one of the above molecules to detect the presence of antibodies. The ability of V_{H}s of the present invention to bind to protein A may be exploited for V_{H} purification and detection in diagnostic tests, immunoblotting and immunocytochemistry.

Libraries of V_{H} domains are also encompassed by the present invention. The V_{H} domain libraries may include a variety of display formats, including phage display, ribosome display, microbial cell display, yeast display, retroviral display, or microbead display formats or any other suitable format.

Analysis of the V_{H}s of the present invention and naturally occurring camelid V_{H}H and shark VNAR single-domain antibodies show analogies in displaying high solubility and reversible thermal unfolding. It is presently found, through analysis of pI (see Example 8), that camelid V_{H}H pools have an abundance of clones with acidic pI (53% acidic versus 43% basic). In germline clones (C. dromedaries), the V_{H} pool is predominantly comprised of V_{H} segments of basic pI, while the opposite is true of the V_{H}H pool, which is predominately populated with V_{H}H segments of acidic pI. It is also presently observed that an overwhelming majority of V_{H} segments (92%) in the human germline V_{H} pool are basic. Thus, a clear correlation has been presently identified between V_{H} solubility and acidic pI; while not all the non-aggregating V_{H}s are acidic, the acidic V_{H}s are non-aggregating. Therefore, the proportion of non-aggregating V_{H}s in a library can be increased by using an acidic scaffold for library construction and/or biasing randomization towards acidic residues and/or against basic ones.

The V_{H} domains and libraries thereof of the present invention may further comprise an acidic amino acid in CDR1, CDR2, and/or CDR3. For example, and without wishing to be limiting, V_{H} domains and libraries thereof may comprise Asp or Glu at position 32 of H1/CDR1, or at other positions in H1/CDR1 or in H1/CDR1, H2/CDR2 or H3/CDR3.

The V_{H} domain and libraries thereof of the present invention may be based on any appropriate V_{H} sequence known in the art. By the term “based on”, it is meant that the V_{H} domain is obtained by the methods of the present invention using a “scaffold” as the initial V_{H} domain. A person of skill in the art would readily understand that, while a V_{H} domain library may be based on a single scaffold, or a number of scaffolds, the CDR/hypervariable loops may be randomized. As such, a large number of V_{H} domains with sequences varying in the randomized regions may be obtained; this is known in the art as a “pool” or “library” of V_{H} domains. The V_{H} domains in the pool V_{H} domains may each recognize the same or different epitopes. Additionally, the scaffolds upon which the V_{H} domains of the present invention are based may possess one or more of the characteristics of non-aggregating V_{H} domains, as described above.

In a particular non-limiting example, the V_{H} domains of the present invention are based on V_{H} sequences having an acidic pI. The V_{H} domains of the present invention may be based on any human germline sequences with acidic pI, in particle those from the V_{H}3 family, and more particularly those with protein A binding activity; for example, but not to be considered limiting, the V_{H} domain may be based on human germline sequence 1-f V_{H} segment, 1-24 V_{H} segment and 3-43 V_{H} segment (see FIG. 14; SEQ ID NOs: 182-184). Alternatively, the V_{H} domain may be based on camelid V_{H} cDNAs or camelid germline V_{H} segments with acidic pIs. The acidic camelid germline V_{H} segments used as library scaffold can be any of those known in the art; in a specific, non-limiting example, the V_{H} segments may be those described in Nguyen et al., 2000. In yet another alternative, the V_{H}s and the libraries thereof presently described may be based on camelid V_{H}H cDNAs or camelid germline V_{H}H segments with acidic pIs. The acidic camelid V_{H} or V_{H}H cDNA or germline sequences used as library scaffold can any of those known in the art; for example, but not limited to those described in Harmsen et al. (2000), Tanha et al. (2002), those in the pool of V_{H}Hs with NCBI Accession numbers AB091838-AB092333, in Nguyen et al. (2000), or those in the VBASE database of human sequences (Medical Research Council, Centre for Protein Engineering).

The V_{H} domain and libraries thereof of the present invention may also be based on a scaffold further comprising an acidic amino acid in CDR1, CDR2, and/or CDR3. In a non-limiting example, the scaffold may comprise Asp or Glu at position 32 of H1/CDR1, or at other positions in H1/CDR1 or in H1/CDR1, H2/CDR2 or H3/CDR3.

The V_{H} domains and libraries thereof of the present invention may further be based on chimeric scaffolds; for example, and without wishing to be limiting, the chimeric scaffolds may comprise one or more camelid or shark CDR/hypervariable loop sequences on human framework sequences. In a specific, non-limiting example, the chimeric scaffold comprises a camelid CDR3/H3 loop on a human V_{H} framework (human CDR1/H1 and CDR2/H2). Chimeric antibody domains are well-known in the art, as are the methods for obtaining them.

In a specific non-limiting example, the present invention provides a V_{H} domain or library thereof, wherein a) the V_{H} domain is based on HVHP430 (SEQ ID NO:1); b) the Cys at positions 99 and 100d of CDR3 are maintained; c) the remaining 14 amino acid residues of CDR3 are randomized; d) amino acid residue 94 is randomized; and e) the 8 amino acid residues of CDR1/H1 are randomized. In a further non-limiting example, there is provided a V_{H} domain library, wherein a) the V_{H} domain is based on HVHP430 (SEQ ID NO:1); b) the amino acid residues at 93-102 (93/94-CDR3) positions are derived from llama V_{H}Hs; c) the 8 amino acid residues of CDR1/H1 are randomized. In yet another non-limiting example, a V_{H} domain or library thereof is provided, wherein a) the V_{H} domain is based on HVHP430 (SEQ ID NO:1); b) the CDR3 comprises a sequence selected from SEQ ID NOs:24-90 and SEQ ID NOs:33-63; c) the 8 amino acid residues of CDR1/H1 are randomized.

The proportion of non-aggregating V_{H}s in the libraries of the present invention, as described above, may be greater than in conventional libraries.

In yet another aspect, the V_{H} domains and libraries thereof of the present invention may be mixed randomized libraries. In this type of library, the CDRs are produced in vitro by using randomized oligonucleotides and methods known in the art.

Using a method of the present invention, non-aggregating, refoldable V_{H}s were isolated in one example. Among these, three had acidic pI and two had a CDR1 Cys residue that formed inter CDR1-CDR3 disulfide linkages. In addition, three V_{H}s with a pair of Cys in their CDR3 (as well as the parent scaffold, HVHP430) formed intra-CDR3 disulfide linkages. However, in one embodiment, the V_{H}s of the present invention comprising non-canonical disulfide linkage spanning CDR1 to CDR3 refold to their native structure more efficiently than those with intra-CDR3 disulfide linkages or only the canonical disulfide bond between Cys22 and at Cys92 during the refolding step of the panning. Therefore, these V_{H}s may be favorably selected during the binding step of the panning. Additionally, most non-aggregating, refoldable V_{H}s have theoretical pIs below 6, possibly due to the fact that above pI 6 (and especially closer to pI 7) V_{H}s become aggregation-prone, as their net charge approaches zero. Among the nine V_{H}s isolated by the heat-denaturation method, three of the four V_{H}s with lowest solubility had a pI around 7.0 (6.4-7.3).

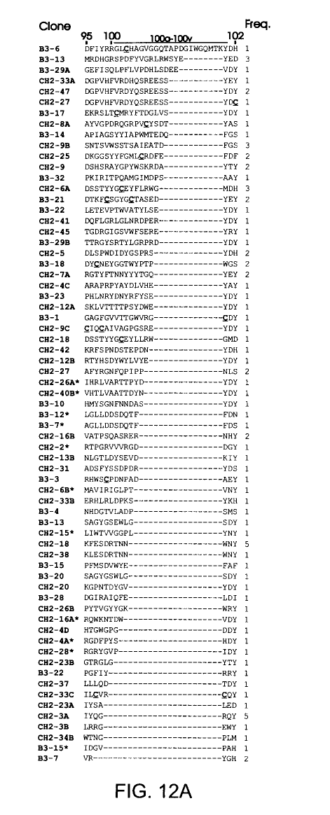

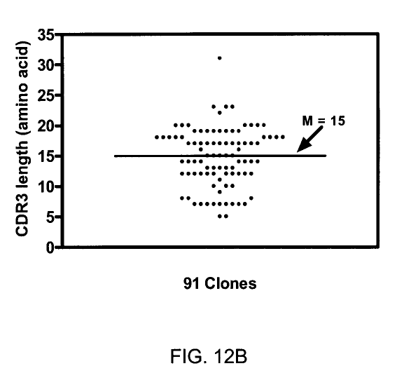

The V_{H} of the present invention may be any V_{H} that exhibits the desired characteristics, as described herein. In a specific, non-limiting example, the human V_{H} domain may comprise one of huVHAm302 (SEQ ID NO:15), huVHAm309 (SEQ ID NO:17), huVHAm316 (SEQ ID NO:19), huVHAm303 (SEQ ID NO:164), huVHAm304 (SEQ ID NO:16), huVHAm305 (SEQ ID NO:15165 huVHAm307 (SEQ ID NO:166), huVHAm311 (SEQ ID NO:167), huVHAm315 (SEQ ID NO:18), huVHAm301 (SEQ ID NO:163), huVHAm312 (SEQ ID NO:168), huVHAm320 (SEQ ID NO:171), huVHAm317 (SEQ ID NO:170), huVHAm313 (SEQ ID NO:169), huVHAm431 (SEQ ID NO:23), huVHAm427 (SEQ ID NO:21), huVHAm416 (SEQ ID NO:20), huVHAm424 (SEQ ID NO:175), huVHAm428 (SEQ ID NO:22), huVHAm430 (SEQ ID NO:176), huVHAm406 (SEQ ID NO:172), huVHAm412 (SEQ ID NO:173) or huVHAm420 (SEQ ID NO:174). In another non-limiting example, the human V_{H} domain or libraries thereof comprises a sequence selected from any of SEQ ID NOS: 101 to 131, or 132-162, or a sequence selected from any of those shown in FIG. 12A (SEQ ID NOs:24-90), or combinations thereof.

The V_{H} domain as described herein may be obtained by the novel methods described below. In a non-limiting example, the V_{H} domain may be isolated from a phagemid-based phage-display library. The use of a fully-synthetic designed phagemid-based phage display library, followed by selection characterized by enrichment for human V_{H}s with the desired properties mentioned herein, is an approach that has not been previously used for human V_{H}s.

In one embodiment, the present invention provides a method of increasing the power or efficiency of selection of non-aggregating V_{H} domains by:
a) providing a phagemid-based V_{H} domain phage-display library, wherein the library is produced by multivalent display of V_{H} domains on the surface of phage; and
b) panning, using the phage-V_{H} domain library and a binding target,
wherein the method comprises a step of selection of non-aggregating phage-V_{H} domains. In one example, the selection step may occur prior to the step of panning and may comprise subjecting the phage-V_{H} domain library to a heat denaturation/re-naturation step. Alternatively, the selection step may occur following panning and may comprise sequencing individual clones to identify the V_{H} with acidic pIs. In another alternative, both the heat denaturation/re-naturation step and the sequencing step are performed.

For example, and without wishing to be limiting, the method of increasing the power or efficiency of selection of non-aggregating V_{H} domains may comprise:
a) providing a phagemid-based V_{H} domain phage-display library, wherein the library is produced by multivalent display of V_{H} domains on the surface of phage;
b) subjecting the phagemid-based V_{H} domain phage-display library to a heat denaturation/re-naturation step; and
c) panning, using the phage-V_{H} domain library and a target.

In another non-limiting example, the method of increasing the power or efficiency of selection of non-aggregating V_{H} domains may comprise:
a) providing a phagemid-based V_{H} domain phage-display library, wherein the library is produced by multivalent display of V_{H} domains on the surface of phage;
b) panning, using the phage-V_{H} domain library and a target; and
c) sequencing individual clones to identify V_{H} domains having an acidic pI,

The method as described above may comprise subsequent rounds of panning; for example, 2, 3, 4, 5, 6, 7, 8, 9, or 10 rounds of panning may be performed. The method as described may also comprise isolation of specific V_{H} domains by amplifying the nucleic acid sequences coding for the V_{H} domains; cloning the amplified nucleic acid sequences into an expression vector; transforming host cells with the expression vector under conditions allowing expression of nucleic acids coding for V_{H} domains; and recovering the V_{H} domains having the desired specificity.

The phagemid-based V_{H} domain phage-display library may be prepared by any method known in the art. For example, and without wishing to be limiting, the library may be prepared by inserting phagemids, each comprising a nucleic acid encoding a V_{H} domain, into a bacterial species; contacting the bacterial species with a hyperphage and subjecting the bacterial species to conditions for infection; and, subjecting the phagemid-inserted and hyperphage-infected bacterial species to conditions for production of a phage-V_{H} domain library.

The “phagemid” used in the method of the present invention is a vector derived by modification of a plasmid, containing an origin of replication for a bacteriophage as well as the plasmid origin of replication. The phagemids comprise the filamentous bacteriophage gIII or a fragment thereof; in this example, the nucleic acid encoding the V_{H} domain is expressed in fusion with the full or truncated gIII product (pIII) and displayed through the pIII on the phage particle. The phagemids also comprise a nucleic acid encoding a V_{H} domain; each phagemid may comprise a nucleic acid encoding various members of a pool of V_{H} domains. The insertion of the phagemids into the bacterial species may be done by any method know in the art.

The V_{H} domain encoded in the phagemids may be based on any appropriate V_{H} sequence. The V_{H} domain scaffold may be any suitable scaffold known in the art. In a particular non-limiting example, the V_{H} domains of the present invention are based on V_{H} sequences having an acidic pI. The V_{H} domains of the present invention may be based on any known human germline sequences with acidic pI, in particle those from the V_{H}3 family, and more particularly those with protein A binding activity; for example, but not to be considered limiting, the V_{H} domain may be based on human germline sequence 1-f V_{H} segment, 1-24 V_{H} segment and 3-43 V_{H} segment (see FIG. 14). Alternatively, the V_{H} domain may be based on camelid V_{H} cDNAs or camelid germline V_{H} segments with acidic pIs. The acidic camelid germline V_{H} segments used as library scaffold can be any of those known in the art; in a specific, non-limiting example, the V_{H} segments may be those described in Nguyen et al., 2000. In yet another alternative, the V_{H}s and the libraries thereof presently described may be based on camelid V_{H}H cDNAs or camelid germline V_{H}H segments with acidic pIs. The acidic camelid V_{H}H cDNA used as library scaffold can any of those known in the art; for example, but not limited to the VH segments may be those described in Harmsen et al. (2000), Tanha et al. (2002), those in the pool of V_{H}Hs with NCBI Accession numbers AB091838-AB092333, or in Nguyen et al. (2000). Various other scaffolds on which the V_{H} domains can be based are described above. As would be recognized by those of skill in the art, while the V_{H} domains in the library may be based on a scaffold, a large number of different V_{H} domains are present in the library due to randomization of selected regions. The proportion of non-aggregating V_{H}s in the library of the present invention may be greater than in conventional libraries.

The phagemid may be inserted into any suitable bacterial species and strain; a person of skill in the art would be familiar with such bacterial species and strains. Without wishing to be limiting, the bacterial species may be, for example, E. coli; in another non-limiting example, the E. coli strain may be TG1, XL1-blue, SURE, TOP10F′, XL1-Blue MRF′, or ABLE® K. Methods for inserting the phagemid into the bacterial species are well known to those in the art.

In the method of the present invention as just described above, the library used is produced by multivalent display of V_{H} domains on the surface of phage. This may be accomplished by contacting the bacterial species, into which the phagemid has been inserted, with a hyperphage and subjecting the bacterial species to conditions for infection.

“Hyperphage” are a type of helper that have a wild-type pIII phenotype and are therefore able to infect F(+) Escherichia coli cells with high efficiency; however, their lack of a functional pIII gene means that the phagemid-encoded pIII-antibody fusion is the sole source of pIII in phage assembly. This results in a considerable increase in the fraction of phage particles carrying an antibody fragment on their surface and leads to phage particles displaying antibody fragments multivalently. In one non-limiting example, the hyperphage may be M13KO7ΔpIII. However, other suitable homologues can be used in the method of the present invention; for example, and without wishing to be limited in any manner, Ex-phage (Baek et al, 2002) or Phaberge (Soltes et al, 2003).

The conditions under which the bacterial species are infected by hyperphage are well known in the art; for example, and without wishing to be limiting in any manner, the conditions may be those described in Arbabi-Ghahroudi, et al. (2008) or Rondot et al. (2001), or any other conditions suitable for infection of the bacteria by the hyperphage.

The infected bacterial species is then submitted to conditions for production of a phage-V_{H} domain library. Such conditions are well known in the art; for example, and without wishing to be limiting, suitable conditions are described in (Arbabi-Ghahroudi, et al., 2008; Harrison, et al., 1996).

In the method of the present invention, panning is performed using the phage-V_{H} domain library and a target. As is known to a person of skill in the art, “panning” refers to a process in which a pool of filamentous phage-displayed antibody libraries (for example, the phage- V_{H} domain library of the present invention) is exposed to the target (or “antigen”) of interest. The target may be either fixed or available, or may be on a solid surface, in solution, on the cell surface, or any other suitable format. The non-binding phage-antibodies may be removed by various methods, including washing extensively with buffer containing detergents such as Tween 20; alternatively, phage bound to a biotinylated target may be captured out by streptavidin magnetic beads. The bound phage-antibodies may then be eluted from the target by methods well-known in the art. The eluted phage-antibodies may then be amplified (propagated) in F+ bacterial host. The process of selection and amplification may be performed in one or more than one round of panning; for example, 2, 3, 4, 5, 6, 7, 8, 9, or 10 rounds of panning may be performed. This results in specific enrichment of antibody-phage binders to the target and leads to the isolation of mono-specific antibody (for instance V_{H} domains). Conditions for panning are well-known to those of skill in the art; for example, the conditions may be those described in Marks et al (1991), Griffiths et al (1994), or Sidhu et al (2004), Hoogenboom (2002), Bradbury (2004) or any other suitable conditions.

The “target” used in the panning step may be any appropriate selected target. For example, the target may be a substantially purified antigen, antigen conjugated to molecules such as biotin or similar molecules, a partially-purified antigen, a cell, a tissue; the target may also be may be either fixed or available, or may be on a solid surface, in solution, on the cell surface, or any other suitable format (see Hoogenboom, 2005). The conjugation of antigen with, for example biotin, make the selection step straightforward and more efficient and required much lower amount of purified antigen. The target may also be selected based on the desired specificity of the resulting phagemid-based V_{H} domain phage-display library or of the V_{H} domains. The target may be any type of molecule of interest; for example, the target may be an enzyme, a cell-surface antigen, TNF, interleukins, molecules in the ICAM family etc. A person of skill in the art would readily understand that the V_{H} domain libraries obtained by the methods described herein can be directed toward any target of interest or of therapeutic importance. For example, and without wishing to be limiting in any manner, the enzyme may be α-amylases, carbonic anhydrases, or lysozymes.

A method of the present invention may further comprises a step of selection of non-aggregating phage-V_{H} domains.

In one embodiment, the selection step may occur prior to the step of panning and may comprise subjecting the phage-V_{H} domain library to a heat denaturation/re-naturation step.

This step involves thermal unfolding of the V_{H} domains, with subsequent refolding to their native conformation, and is undertaken by any method know in the art; see for example Jespers et al (2004). For example, and without wishing to be limiting, the phage-V_{H} domain library may be subjected to denaturation at a temperature in the range of about 55° C. to about 90° C.; the temperature may be 55, 60, 65, 70, 75, 80, 85, or 90° C., or any temperature therebetween. In one embodiment, the phage-V_{H} domain library is maintained at this elevated temperature for a time in the range of about 1 minute to about 30 minutes; for example and without wishing to be limiting, the temperature may be maintained for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 minutes, or any time therebetween. Subsequently, phage-V_{H} domain library is subjected to renaturation by returning the temperature to a lower temperature, for example room temperature or lower, 4 or 5° C., for an amount of time similar to that used for denaturation. A person of skill in the art will recognize that the temperature at which the V_{H}s in the phage-V_{H} domain library denature will depend on the nature of the V_{H} domain(s) and their melting temperature. Furthermore, the skilled person will understand that, in some embodiments, higher denaturation temperatures may be combined with shorter exposure times; similarly, in other embodiments, lower denaturation temperatures may be combined with longer exposure times. The denaturation/renaturation step may be performed in any appropriate aqueous buffer know in the art; for example, and without wishing to be limiting in any manner, the buffer may be a Tris buffer, PBS buffer, HEPES buffer, carbonate buffer, or water.

In another embodiment, the method may comprise the step of sequencing individual clones to identify V_{H}s with acidic pIs. This screening step of non-aggregating V_{H} domains is based on theoretical pI values, which may be determined by any method known in the art. For example, and without wishing to be limiting, the theoretical pIs may be determined by commercially available software packages. As described previously, the present invention has shown that V_{H} having an acidic pI may be soluble and non-aggregating. Screening non-aggregating V_{H} domains from among the aggregating V_{H}s based on pI values obtained simply by DNA sequencing avoids the need for subcloning, expression, purification and biophysical characterization of a large number of V_{H}s.

In a further embodiment, both the heat denaturation/re-naturation step and the sequencing step are performed.

The method as described herein may also comprise isolation of specific V_{H} domains by amplifying the nucleic acid sequences coding for the V_{H} domains in the recovered phage-V_{H} domains; cloning the amplified nucleic acid sequences into an expression vector; transforming host cells with the expression vector under conditions allowing expression of nucleic acids coding for V_{H} domains; and recovering the V_{H} domains having the desired specificity. Methods and specific conditions for performing these steps are well-known to a person of skill in the art.

The method as described above is a novel combination of using a phagemid-vector based phage-display produced by the use of hyperphage and a selection step based on heat denaturation or analysis of theoretical pIs. This novel method can increase the efficiency for selection of non-aggregating human V_{H}s. In a non-limiting example, the present method may select V_{H} domains comprising non-canonical disulfide bonds, as described above; without wishing to be limiting, the non-canonical disulfide bonds may occur in CDR1 and/or CDR3. In another example, the method as described above may select V_{H} domains with acidic pIs.

Compared to phage vector-based systems, the phagemid vector-based phage display system of the present method provides many advantages including: ease of constructing large libraries which is desirable in the case of non-immune libraries; suitability for isolating high affinity binders from immune or affinity maturation libraries; ease of manipulation for improving affinity or biophysical properties; and facile switching from antibody-pIII fusion to un-fused antibody fragments for rapid antibody expression and analysis. In addition, use of helper phages that result in a multivalent display (Rondot et al., 2001; Baek, H. et al., 2002; Soltes, G. et al., 2003), e.g., hyperphage (M13KO7ΔpIII) in the method of the present invention provides the advantages afforded by the phage vector-based display systems (due to the avidity effect), including: high yield of binders and fewer rounds of panning (O'Connell et al., 2002); a more efficient enrichment of antibodies for cell-surface antigens; and suitability for selecting antibodies to cell surface receptors that require self-cross linking (Becerril et al., 1999; Huie et al., 2001). Moreover, with the phagemid vector system, switching between monovalent and multivalent formats can be readily made by using the appropriate type of helper phage (Rondot et al., 2001; O'Connell et al., 2002; Kirsch et al., 2005). In order to further leverage the advantages phagemid-based libraries offer in terms selecting for non-aggregating V_{H}s, we decided to employ hyperphage technology (Rondot et al.,2001) to adapt the heat denaturation strategy described above (Jespers et al., 2004) to phagemid-based libraries.

In yet another embodiment, the present invention provides a method of increasing the power or efficiency of selection of non-aggregating V_{H} domains by, comprising:
a) providing a phage vector-based V_{H} domain phage-display library, wherein the library is produced based on a V_{H} domain scaffold having an acidic pl;
b) panning, using the phage-V_{H} domain library and a target; and
c) sequencing individual clones to identify V_{H} domains having an acidic pI

The phage vector-based V_{H} domain phage-display library may be prepared by any method known in the art. For example, and without wishing to be limiting, the library may be prepared by inserting phage vectors, each comprising a nucleic acid encoding a V_{H} domain, into a bacterial species; and, subjecting the phage vector-inserted bacterial species to conditions for production of a phage-V_{H} domain library.

A “phage vector” refers to a vector derived by modification of a phage genome, containing an origin of replication for a bacteriophage, but not one for a plasmid; the phage vector may or may not have an antibiotic resistance marker.

The methods for producing a phage vector-based phage-display library are well-established in the art, and would be well-known to the skilled person.

The method as described herein may also comprise isolation of specific V_{H} domains by amplifying the nucleic acid sequences coding for the V_{H} domains in the recovered phage-V_{H} domains; cloning the amplified nucleic acid sequences into an expression vector; transforming host cells with the expression vector under conditions allowing expression of nucleic acids coding for V_{H} domains; and recovering the V_{H} domains having the desired specificity. Methods and specific conditions for performing these steps are well-known to a person of skill in the art.

The present invention is also directed to V_{H}s of the present invention that are fused to a cargo molecule. As used herein, a “cargo molecule” refers to any molecule for the purposes of targeting, increasing avidity, providing a second function, or otherwise providing a beneficial effect. The cargo molecule(s) may have the same or different specificities as the V_{H}s of the invention. For example, and without wishing to be limiting, the cargo molecule may be: a toxin, an Fc region of an antibody, a whole antibody, or enzyme as in the context of antibody-directed enzyme pro-drug therapy (ADEPT) (Bagshawe, 1987: 2006); one or more than one single domain such as V_{H}, V_{L}, V_{H}H, VNAR, etc with the same or different specificities; a liposome for targeted drug delivery; a therapeutic molecule, a radioisotope; or any other molecule providing a desired effect. Methods of coupling or attaching a cargo molecule to a VH domain of the present invention are well-known to those skilled in the art.

The methods and V_{H} domain libraries of the present invention need not be limited to phage-display technologies, but may also be extended to other formats. For example, and without wishing to be limiting, the methods and V_{H} domain libraries of the present invention may be ribosome and mRNA display, microbial cell display, retroviral display, microbead display, etc. (see Hoogenboom, 2005). Conditions for performing these types of display methods are well-known in the art.

The V_{H}s of the present invention may also be recombinantly produced in multimeric form; in a non-limiting example, the V_{H}s may be produced, as dimers, trimers, pentamers, etc. Presentation of the V_{H}s of the present invention in multimeric form(s) may increase avidity of the V_{H}s. The monomeric units presented in the multimeric form may have the same or different specificities.

The present invention further encompasses nucleic acids encoding the V_{H}s of the present invention. As used herein, a “nucleic acid” or “polynucleotide” includes a nucleic acid, an oligonucleotide, a nucleotide, a polynucleotide, and any fragment, variant, or derivative thereof. The nucleic acid or polynucleotide may be double-stranded, single-stranded, or triple-stranded DNA or RNA (including cDNA), or a DNA-RNA hybrid of genetic or synthetic origin, wherein the nucleic acid contains any combination of deoxyribonucleotides and ribonucleotides and any combination of bases, including, but not limited to, adenine, thymine, cytosine, guanine, uracil, inosine, xanthine and hypoxanthine. The nucleic acid or polynucleotide may be combined with a carbohydrate, a lipid, a protein, or other materials. A nucleic acid sequence of interest may be chemically synthesized using one of a variety of techniques known to those skilled in the art, including, without limitation, automated synthesis of oligonucleotides having sequences which correspond to a partial sequence of the nucleotide sequence of interest, or a variation sequence thereof, using commercially-available oligonucleotide synthesizers, such as the Applied Biosystems Model 392 DNA/RNA synthesizer.

The nucleic acids of the V_{H}s of the present invention may be comprised in a vector. Any appropriate vector may be used, and those of skill in the art would be well-versed on the subject.

The present invention also provides host cells comprising the nucleic acid or vector as described above. The host cell may be any suitable host cell, for example, but not limited to E. coli, or yeast cells. Non-limiting specific examples of suitable E. coli strains are: TG1, BL21(DE3), and BL21(DE3)pLysS.

The V_{H} domains of the present invention may possess properties that are desirable for clinical and diagnostic applications. In one embodiment, the V_{H}s may be labelled with a detectable marker or label. Labelling of an antibody may be accomplished using one of a variety of labelling techniques, including peroxidase, chemiluminescent labels known in the art, and radioactive labels known in the art. The detectable marker or label of the present invention may be, for example, a non-radioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker or label may be a radioactive marker, including, for example, a radioisotope. The radioisotope may be any isotope that emits detectable radiation. Radioactivity emitted by the radioisotope can be detected by techniques well known in the art. For example, gamma emission from the radioisotope may be detected using gamma imaging techniques, particularly scintigraphic imaging. In addition, detection can also be made by fusion to a green fluorescent protein (GFP), RFP, YFP, etc.

The V_{H}s of the present invention may also be used in a high-throughput screening assay, such as microarray technology, in which the use of the V_{H} domain is advantageous or provides a useful alternative compared to conventional IgG.

In another aspect, the invention provides a pharmaceutical composition comprising one or more than one V_{H}s in an effective amount for binding thereof to an antigen, and a pharmaceutically-acceptable excipient. Appropriate pharmaceutical excipients are well-known to those of skill in the art.

In a further embodiment, the invention provides a method of treating a patient, comprising administering a pharmaceutical composition comprising one or more V_{H}s to a patient in need of treatment. For those in the art, it is apparent that libraries such as those disclosed herein may be a source of binders to targets. Therefore, they can be used for therapy, diagnosis and detection. Indications that can be targeted by V_{H} domains of the present invention are cancer (for detection of tumor markers and/or treatment of any cancer), inflammatory diseases (which include killing the target cells, blocking molecular interactions, modulating target molecules by antibodies), autoimmune diseases (for example, lupus, rheumatoid arthritis etc.), neurodegenerative diseases (for example Parkinson's disease, Alzheimer's disease, etc) infectious disease caused by prion, viral, bacterial and fungi agents or, in general, any infectious disease resulted from infection by any known or unknown microorganism or agent. Targets may include any molecules that are specific to a given disease state. For example, and not wishing to be limiting in any manner, the targets may include: cell-surface antigens, enzymes, TNF, interleukins, molecules in the ICAM family etc. The libraries obtained in accordance to the present invention may also be used to obtain V_{H} domains for detecting pathogens. Pathogens can include human, animal or plant pathogens such as bacteria, eubacteria, archaebacteria, eukaryotic microorganisms (e.g., protozoa, fungi, yeasts, and moulds), prions, viruses, and biological toxins (e.g., bacterial or fungal toxins or plant lectins). A person of skill in the art would readily understand that the V_{H} domain libraries obtained by the methods described herein can be directed toward any target of interest. In a non-limiting example, the target may be an enzyme; in a further example, and without wishing to be limiting, the enzyme may be lysozymes, α-amylases or carbonic anyhdrases.

In yet another aspect, the invention contemplates the provision of a kit useful for the detection and determination of binding of one or more than one V_{H} to a particular antigen in a biological sample. The kit comprises one or more than one V_{H} and one or more reagents. The one or more than one V_{H} domain may be labelled. Additionally, the kit may also comprise a positive control reagent. Instructions for use of the kit may also be included.

The V_{H} domains of the present invention may also be used in antibody microarray technology. This technology is an alternative to traditional immunoassays, and many thousands of assays can be run in parallel. Antibody V_{H} domains are favoured over whole IgG in this type of assay since they are small, stable and highly specific reagents. Methods for antibody microarrays are well-known in the art.

The present invention will be further illustrated in the following examples. However, it is to be understood that these examples are for illustrative purposes only and should not be used to limit the scope of the present invention in any manner.

### EXAMPLES

Unless indicated otherwise, molecular biology work was done using standard cloning techniques (Sambrook et al., 1989). Phagemid pHEN4 (Arbabi-Ghahroudi et al., 1997) was modified by introducing a second non-compatible Sfi I site and six His codons. The new vector designated pMED1 was used for phage display library construction. pSJF2H plasmid was used for soluble expression of single domain antibodies in E. coli. pSJF2H is identical to pSJF2 (Tanha et al., 2003), except that it expresses proteins in fusion with His₆ instead of His₅.

### Example 1

### HVHP430 V_{H} Library Construction

A fully-synthetic, phagemid-based human V_{H} phage display library was constructed.

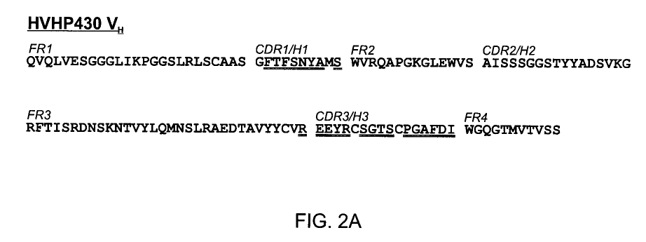

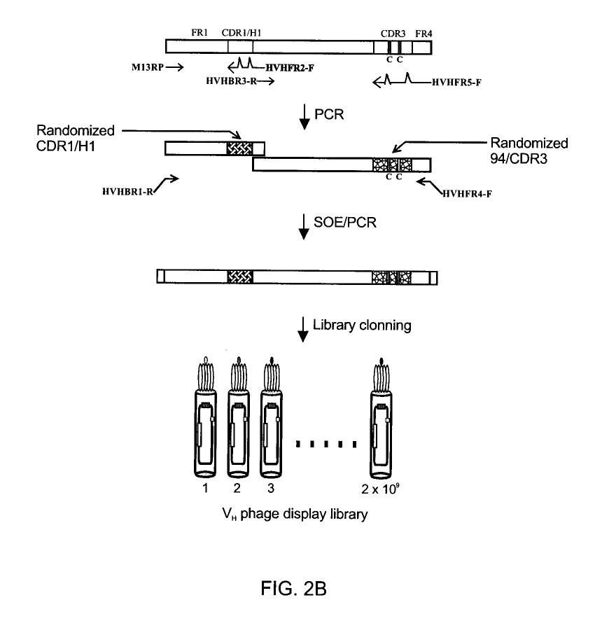

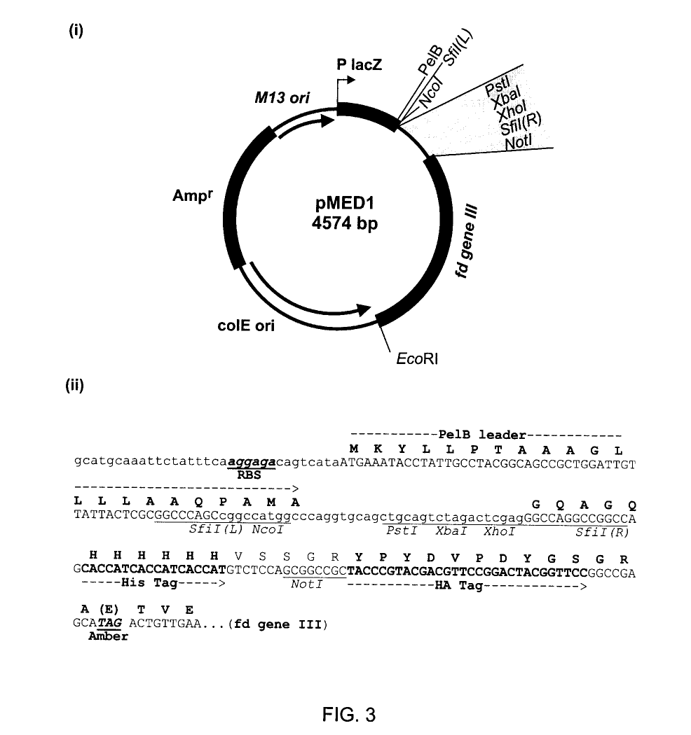

In constructing the V_{H} library on the HVHP430 scaffold (FIG. 2A, SEQ ID NO:1), the two CDR3 Cys were maintained to promote the formation of intra-CDR disulfide linkage and thus, to increase the frequency of enzyme-inhibiting V_{H}s in the library. The remaining 14 CDR3 positions, position 94 as well as eight H1/CDR1 positions were randomized (FIG. 2A). CDR2 was left untouched as it has been shown to be involved in protein A binding (Randen et al., 1993; Bond et al., 2003). Besides, CDR2-lacking VNARs (Stanfield et al., 2004) or camelid V_{H}Hs utilizing their CDR1 and CDR3 (Decanniere et al., 1999) or just CDR3 (Desmyter et al., 2001) for antigen recognition demonstrate nanomolar affinities. The library was constructed with a phagemid vector (FIG. 3) according to the scheme shown in FIG. 2B.

The human V_{H} HVHP430 (To et al., 2005), which has two Cys residues in its CDR3 at position 99 and 100d, was used as the framework to construct a library by randomizing residues in CDR1 and CDR3 and position 94. Using a plasmid containing the HVHP430 gene as template and the primer pairs HVHBR1-R/HVHFR2-F and HVHBR3-R/HVHFR5-F (see Table 1 for listing of primers used), two overlapping fragments with randomized H1/CDR1 and 94/CDR3 codons, respectively, were constructed by standard polymerase chain reactions (PCRs).

TABLE 1
List of the primers used for V_{H} clonings.

| Designation | Sequence |
|---|---|
| HVHBR1-R | 5′- |
| (SEQ ID NO: 4) | CATGTGTAGACTCGCGGCCCAGCCGGCCATGGCCCAGGTGCAGCT |
| | GGTGGAGTC-3′ |
| HVHFR2-F | 5′-GAGCCTGGCGGACCCAGSYCATANHSTNAKNGNTAANSNTAWM |
| (SEQ ID NO: 5) | TCCAGAGGCTGCACAGGAG-3′ |
| HVHBR3-R | 5′-TGGGTCCGCCAGGCTCCAGGGAAG-3′ |
| (SEQ ID NO: 6) | |
| HVHFR5-F | 5′-TGAAGAGACGGTGACCATTGTCCCTTGGCCCCAADASBNMNNM |
| (SEQ ID NO: 7) | NNMNNMNNGCAMNNMNNMNNMNNACAMNNMNNMNNMNNWSY |
| | CACACAGTAATACACAGCCGT-3′ |
| HVHFR4-F | 5′-CATGTGTAGATTCCTGGCCGGCCTGGCCTGAAGAGACGGTGACC |
| (SEQ ID NO: 8) | ATTGTCC-3′ |
| HVHP430Bam | 5′-TTGTTCGGATCCTGAAGAGACGGTGACCAT-3′ |
| (SEQ ID NO: 9) | |
| HVHP430Bbs | 5′-TATGAAGACACCAGGCCCAGGTGCAGCTGGTGGAGTCT-3′ |
| (SEQ ID NO: 10) | |
| M13 RP | 5′-CAGGAAACAGCTATGAC-3′ |
| (SEQ ID NO: 11) | |

The PCR products were run on a 1% agarose gel and the sub-fragments were gel-purified using the QIAquick Gel Extraction™ kit (QIAGEN Inc., Mississauga, ON, Canada). The sub-fragments were spliced and subsequently amplified by splice overlap extension-PCR (Aiyar et al., 1996), using HVHBR1-R and HVHFR4-F primers. The constructed V_{H} products were purified using the QIAquick PCR Purification™ kit (QIAGEN Inc.) and digested with Sfi I restriction endonuclease. In parallel, pMED1 phagemid vector was cut with Sfi I restriction endonuclease, and then with Pst I and Xho I and the linearized vector was purified using QIAquick PCR Purification™ kit. Ligation and transformations were performed (Arbabi-Ghahroudi et al., 2009). Ligation was performed in a total volume of 1 mL with a 1:1.5 molar ratio of vector to insert using a total of 84 μg vector and 11 μg of V_{H} insert and the ligated mixture was desalted prior to transformation using QIAquick PCR Purification™ kit. A total of 105 transformations were performed by mixing 50 μL of TG1 cells with 1 μL of the ligated product. The library was amplified and stored frozen. The functional size of the library was determined. Library phage production was performed according to Arbabi-Ghahroudi et al. (2009) except that 5×10¹⁰ library cells were used to inoculate a 500 mL 2×YT/Amp/1% glucose medium and the overnight phage amplification was performed in 500 mL instead of 300 mL of the recommended medium. The V_{H}s are in frame with PeIB leader peptide on their N-termini and His₆-tag, HA-tag, amber stop codon and fd gene III on their C-termini. A monovalent display of V_{H} on the surface of phage is based upon using the helper phage M13KO7 for superinfection.

DNA sequencing of a library sample (n=36) revealed unique clones with mutations at the intended positions and no deleted V_{H} varieties.

### Example 2

### Panning and Phage ELISA

### A. Panning in a Monovalent Display Format

In the first panning attempt, the helper phage M13KO7 was used for super-infection, resulting in a monovalent display of V_{H}s on the surface of the phage (O'Connell et al., 2002). The initial aim was to explore the feasibility of the library in yielding enzyme inhibitors. Four rounds of panning were performed against α-amylase, lysozyme and carbonic anhydrase, as described below.

A total of 50 μg antigen (lysozyme, α-amylase and carbonic anhydrase) in 100 μL PBS was used to coat Maxisorp™ wells (Nunc, Roskilde, Denmark) overnight at 4° C. The solutions were then removed and the wells were blocked by adding 200 μL of 3% BSA in PBS and incubating the wells for 2 h at 37° C. The blocking reagent was removed and 10¹² library phage (input) was added to each well, and the wells were incubated for 2 h at 37° C. The supernatants were removed and wells were washed 7 times with 0.1% PBST (0.1% v/v Tween 20 in PBS). To elute the bound phage, 100 μL triethylamine (100 mM in H₂O, made fresh daily) was added to each well followed by incubation at room temperature for 10 min. To neutralize the phage solution, the eluted phages were transferred to a tube containing 100 μL 1 M Tris-HCl buffer pH 7.5 and mixed. The phages were used to infect 2 mL of exponentially growing TG1 bacterial cells in LB medium for 15 min at 37° C. (Arbabi-Ghahroudi et al., 2009). Two μL of the infected cells was removed to determine the titer of the eluted phage (output) and to the remainder, 6 mL of 2×TY was added. Ampicillin was added at a final concentration of 50 μg/mL and the culture was incubated at 37° C. for 1 h at 220 rpm. The cells were superinfected by adding M13KO7 helper phage or hyperphage at a 20:1 phage-to-cell ratio and incubating the mixture at 37° C. for 30 min without shaking then for 1.5 h with shaking. The cells were transferred to a flask containing 92 mL of 2×TY medium. Ampicillin and kanamycin were added to a final concentration of 100 and 50 μg/mL, respectively, and the culture was incubated at 37° C. overnight at 250 rpm. Phage was purified and titered (Arbabi-Ghahroudi et al., 2009) and used as input for the second round of panning. For the second, third and fourth rounds of panning, a total of 40, 30 and 20 μg of antigen, respectively, were used. The input phage was the same for all rounds but the number of washes was increased to 9× for the second round, 12x for the third round and 15x for the fourth round.

Sequencing of 80 clones from various rounds showed a predominant enrichment for Gly at positions 35, most likely due to the favorable biophysical properties Gly35 confers to V_{H}s (Jespers et al., 2004a). However, over 40% of the V_{H}s had amber stop codon (TAG), almost exclusively at CDR1 position 32. The amber stop codon is read as Glu in the phage host, E. coli TG1 (see below).

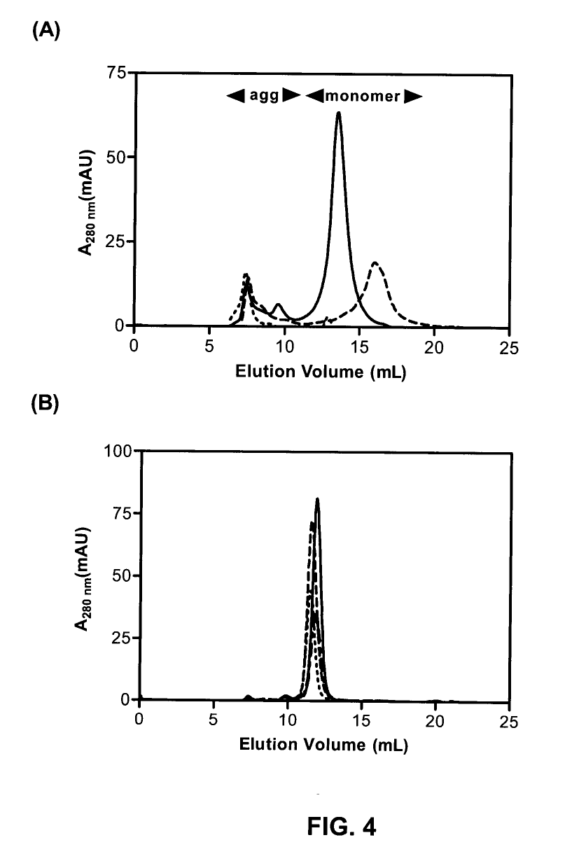

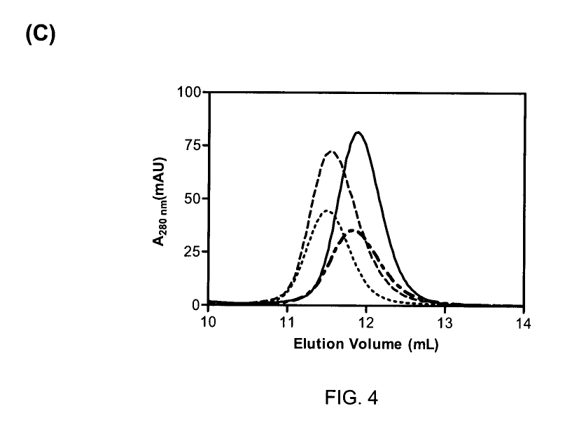

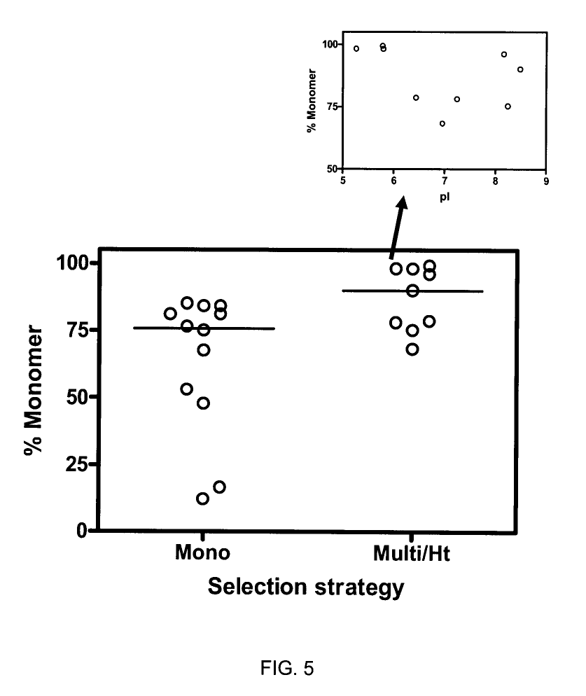

Following panning, 10-20 round 4 clones were tested for binding to their target antigens by phage ELISA (Arbabi-Ghahroudi et al., 2009); 6/20, 10/10 and 19/20 were positive for binding to lysozyme, α-amylase and carbonic anhydrase, respectively. Twelve V_{H}s (three α-amylase binders, four lysozyme binders and five carbonic anyhdrase binders) were sub-cloned into a vector, expressed in 1 L cultures and subjected to Superdex™ 75 gel filtration chromatography for examining their aggregation states. None of the V_{H}s were completely monomeric, ranging from as low as 12% and 16% monomeric to 85% at best (median: 78%) (FIGS. 4A and 5). Additionally, several V_{H}s precipitated at 4° C., not long after their purification.

B. Panning in a Multivalent Display Format with Heat Denaturation

The results of panning with the V_{H} phage display library demonstrated that a selection based solely on binding was not efficient in yielding functional binders. A heat denaturation approach, previously shown to efficiently yield non-aggregating binders from V_{H} phage display libraries (Jespers et al., 2004a), was used. The method was shown to work with a phage vector-based library because of its multivalent presentation but not with a phagemid-based library with a monovalent display format. Thus, to have the phagemid-based phage display library in a multivalent display format, hyperphage, rather than helper phage, was used for superinfection (Rondot et al., 2001).

For selection by heat denaturation, input phage in a multivalent display format was used (i.e., the phages were produced by using hyperphage for superinfection). The input phage was heated at 80° C. for 10 min, cooled at 4° C. for 20 min, centrifuged at maximum speed for 2 min in a microfuge and the supernatant was added to antigen-coated wells for binding. Three rounds of panning against α-amylase by the heat denaturation method were performed. A non-treatment panning was also carried out in parallel as control. Following three rounds of panning, for each condition twenty clones were tested by phage ELISA and all were found to bind to α-amylase (data not shown). Monoclonal Phage ELISA on single colonies was performed (Arbabi-Ghahroudi et al., 2009). ELISA-positive clones were subjected to DNA sequencing to identify their V_{H}s (Arbabi-Ghahroudi et al., 2009). Isoelectric points, pIs, of the V_{H}s were determined using the software Laser gene v6.0 (DNASTAR, Inc., Madison, Wis.). There is minor variance between pI values obtained here (higher by 2%) and those reported elsewhere (Jespers et al., 2004a).

All forty clones were subjected to DNA sequencing, revealing no sequence overlaps between the treatment and non-treatment V_{H}s. Except for two V_{H}s, which were among the non-heat-treatment clones, the remaining V_{H}s had non-Ser residues, predominantly Gly at position 35. Additionally, all V_{H}s had amber stop codons in their CDR1 (position 32) and/or CDR3 and as before, amber codons were predominantly at position 32. In contrast to the non-treatment panning, which similar to the one in the monovalent display format did not yield repeating clones, the panning under heat denaturation yielded V_{H}s which occurred more than once (Table 2, huVHAm302, huVHAm309, huVHAm316), suggesting a non-randomness character of the selection under heat denaturation. Panning was continued only for the one under heat denaturation. Twenty-seven ELISA-positive clones from rounds 4 were sequenced and out of the nine newly identified V_{H}s, eight had amber stop codons (Table 2). Interestingly, for all the round 3 and four clones position 32 if not occupied by an amber codon contained either Asp (D) or Glu (E), suggesting the importance of acidic residues at position 32 for V_{H} stability and non-aggregation. Biased enrichments for binders (scFvs) with amber codons have been observed with other synthetic libraries (Marcus, W. D. et al., 2006a) (Marcus, W. D. et al., 2006b) (Yan, J. P. et al., 2004). A reduced expression of the V_{H}s with amber codons in E. coli TG1 compared to those without should give the phages displaying such V_{H}s growth advantage, leading to their preferential selection.

Selection was characterized by enrichment for V_{H}s with (i) disulfide forming cysteine (Cys) in their CDRs and (ii) acidic isoelectric points (pI). After the third round of panning, the library was enriched for V_{H}s which had acidic pIs and/or an even number of Cys residues in their CDRs where one CDR1 Cys was matched with one or three CDR3 Cys residues (Table 2). Furthermore, either one Cys is missing from or added to the two fixed CDR3 Cys residues to, together with the CDR1 Cys, keep the total number of Cys two or four, respectively. Very frequently camelid and shark single domains have non-canonical Cys residues which almost invariantly come in pairs to form disulfide linkages, many between CDR1 and CDR3. Strong selection for the above two properties is further underlined by the fact that none of the 36 V_{H}s sequenced from the unpanned library had acidic pI or paired Cys residues in their CDR1 and CDR3.

### Legend for Table 2:

Asterisks in CDR1 and 3 denote the amber stop codon which is read as Glu (E) in the phage host, E. coli TG1.
#Mutations in FRs were observed.
†Theoretical pI.
‡Thermal refolding efficiency.
Nd, not determined

TABLE 2
Characteristics of anti-α-amylase VHs isolated from the VH phage display
library by the heat denaturation panning method

| | | | | | | Monomer/ | Inter/intra-V_{H} | | |
|---|---|---|---|---|---|---|---|---|---|
| | | | Frequency | | | protein A | disulfide | TRE^{‡} (%) | |
| V_{H} | H1/CDR1 | Position 94/CDR3 | R3 | R4 | pI^{†} | binding | linkage | 0.5 μM | 5 μM |
| huVHAm302 | DTVSD*SMT | T/DNRSCQTSLCTSTTRS | 5 | 9 | 7.3 | x/✓ | ✓/✓ | | |
| huVHAm309^{#} | VNFSN*GMA | T/AQRACANSPCPGSITS | 2 | 0 | 8.2 | ✓/✓ | x/✓ | 94 | 93 |
| huVHAm316 | DRFTY*SMG | A/LETACTRPACAHTPRF | 2 | 0 | 8.5 | x/✓ | ✓/nd | | |
| huVHAm303 | FRFSYEVMG | T/PKVDC*THPCRERPYF | 1 | 0 | 8.5 | | | | |
| huVHAm304 | FSFSD*GMA | R/LPKQCTSPDCET*VSS | 1 | 0 | 5.3 | ✓/✓ | x/✓ | 99 | 87 |
| huVHAm305 | YRFNN*VMG | T/STPACNQDKCERWRPS | 1 | 0 | 8.8 | | | | |
| huVHAm307 | FSVSD*DMG | T/PLPKCTNPNCKSPPKY | 1 | 0 | 8.2 | | | | |
| huVHAm311 | FRVTPECMT | R/HEVECPT*QCPFHCPS | 1 | 0 | 7.7 | | | | |
| huVHAm315^{#} | DMFSS*GMA | A/APTTCTSHNCAEPFRS | 1 | 0 | 7.0 | x/✓ | ✓/nd | | |
| huVHAm301 | FRISHEGMG | A/YN*ECTKPSCHTKARS | 1 | 0 | 8.8 | | | | |
| huVHAm312 | VMG | A/P*TQCSEGRCLGTASS | 1 | 0 | 8.2 | | | | |
| huVHAm320 | YSVSD*SMG | T/TDPLGAKGQ | 1 | 0 | 8.0 | | | | |
| huVHAm317 | YMISD*IMA | A/PNRAKGQ | 1 | 0 | 8.9 | | | | |
| huVHAm313 | FRFID*DMG | A/GAKGQ | 1 | 0 | 8.5 | | | | |
| huVHAm431 | YTVSSECMG | R/DSKNCHDKDCTRPYCS | 0 | 6 | 8.3 | x/✓ | ✓ | | |
| huVHAm427 | VTLSPECMA | S/CEG*NAF | 0 | 4 | 6.4/ | x/✓ | x/nd | | |
| huVHAm416 | VSFTDDCMA | A/DHTQCRQPEC*SQLCS | 0 | 2 | 5.8 | ✓/✓ | x/✓ | 100 | 97 |
| huVHAm424^{#} | DRVIS*CMG | A/LPPEVCEADVPDRGDL | 0 | 1 | 4.8 | | | | |
| huVHAm428^{#} | FSLSDDCMG | T/GNQACKH*PWPDEALL | 0 | 1 | 5.8 | ✓/✓ | x/✓ | 89 | 86 |
| huVHAm430 | DRVSP*DMA | T/SGVPSGSF | 0 | 1 | 6.5 | | | | |
| huVHAm406^{#} | FSFTP*CMG | G/HKNNC | 0 | 1 | 8.5 | | | | |
| huVHAm412^{#} | DMLSA*CMG | A/KPYHC | 0 | 1 | 8.2 | | | | |
| huVHAm420^{#} | DRFSY*DMA | A/TEESCPEGNCPPPRRS | 0 | 1 | 5.0 | | | | |

The enriched pool of V_{H}s contained a proportion of aggregating V_{H}s. This is not unexpected since CDRs can affect V_{H} solubility (Jespers et al., 2004a; Jespers et al., 2004b; Martin et al., 1997; Desmyter et al., 1996; Decanniere et al., 1999; Vranken et al., 2002). A stable scaffold for library construction was used since it tolerates destabilizing mutations, thus accepting a wider range of beneficial mutations without losing its native fold (Bloom et al., 2006). It has been shown that a library constructed from a stable version of cytochrome P450 BM3 yielded three times more mutants with new or improved enzymatic activity compared to those built on a marginally stable version (Bloom et al., 2006). Similarly, a library constructed with a V_{H} scaffold with improved solubility and stability yielded functional binders against several antigens whereas a library of identical size built on the aggregation-prone wild type version yielded only nonfunctional, truncated V_{H}s (Tanha et al., 2006). In both studies, the library members based on more stable scaffolds were more likely to fold than the ones based on less stable ones. Differences in scaffold stability may account for the fact that the prior art has isolated only one functional V_{H} against human serum albumin from a V_{H} phage display library (Jespers et al., 2004a) compared to several isolated using the method of the present invention from a V_{H} library with 10-fold less diversity. The comparative yield becomes even more significant considering that a subset of the library was tapped into, that with amber-containing V_{H}s, for obtaining binders.

The present library failed to yield soluble binders when panned in a monovalent display format. However, when panned in a multivalent display format by using hyperphage for superinfection and heat denaturation, the library surprisingly yielded non-aggregating V_{H}s. Use of hyperphage is contrary to the prior art, which typically teaches the use of helper phages such as VCS leading to monovalent-display libraries (Vieira et al., 1987; Vaughan et al., 1996; Baca et al., 1997; Hoogenboom et al., 1991).

### Example 3

### Analysis of Clones

Nine V_{H}s, huVHAm302, huVHAm304, huVHAm309, huVHAm315, huVHAm316, huVHAm416, huVHAm427, huVHAm428 and huVHAm431, were identified for subcloning and further analysis. However, all except one (huVHAm431) had amber stop codon which would impede their expression even in an amber suppressing strain such as TG1 which was to be used as the expression host (in TG1 cells, the amber stop codon is read as an amino acid but mostly as a stop codon). Thus, the amber codons were replaced with a non-stop codon that would code for the same amino acid and re-express the resultant V_{H}s.

However, in selecting the appropriate replacement codon, inconsistent information was found with regards to the nature of the amino acids being coded by the amber codon in E. coli TG1 cells. Some have reported Glu as the overwriting amino acids (Hoogenboom, H. R. et al., 1991), (Baek, H. et al., 2002) while others Gln (Soltes, G. et al., 2003) (Marcus, W. D. et al., 2006a). As an exact amino acid designation was essential in terms of avoiding possible disruption of antigen-antibody interactions and not reaching erroneous conclusions in the V_{H} pI analysis (see Example 8), the nature of the amino acid(s) being coded by the amber codon was determined.

To this end, the eight V_{H}s which had amber codons were subcloned in TG1 cells for subsequent amino acid determination by mass spectrometry. However, only one V_{H}, huVHAm302, was produced in sufficient quantity for mass spectrometry analysis.

A 60 μL solution of huVHAm302 at 50 ng/μL in 50 mM ammonium bicarbonate was reduced with 100 μL of 2 mM DTT at 37° C. for 1 h and alkylated with 40 μL of 50 mM iodoacetamide at 37° C. for 30 min. The reagents used for reduction and alkylation were removed by centrifugal ultrafiltration (3,000 MWCO). The protein solution (0.25 mL in 50 mM ammonium bicarbonate) was incubated at 37° C. for 16 h after addition of 1 μL of trypsin solution (0.33 μg/μL). An aliquot of the tryptic digest of huVHAm302 was re-suspended in 10 μL of 0.2% formic acid (aq) and analyzed by nano-reversed-phase HPLC mass spectrometry (nanoRPLC-MS) using a CapLC™ capillary liquid chromatography system coupled to a Q-TOF Ultima™ hybrid quadrupole/time of flight mass spectrometer (Waters, Millford, Mass.) with DDA. The peptides were first loaded onto a 300 μm i.d.×5 mm C18 PepMap100™ trap (LC Packings, San Francisco, Calif.), then eluted off to a Picofrit™ column (New Objective, Woburn, Mass.) using a linear gradient from 5% to 42% solvent B (acetonitrile, 0.2% formic acid) in 23 min, 42% -95% solvent B in 3 min. Solvent A was 0.2% formic acid in water. The peptide MS/MS spectra were searched against the protein sequence using the Mascot™ database searching algorithm (Matrix Science, London, UK).

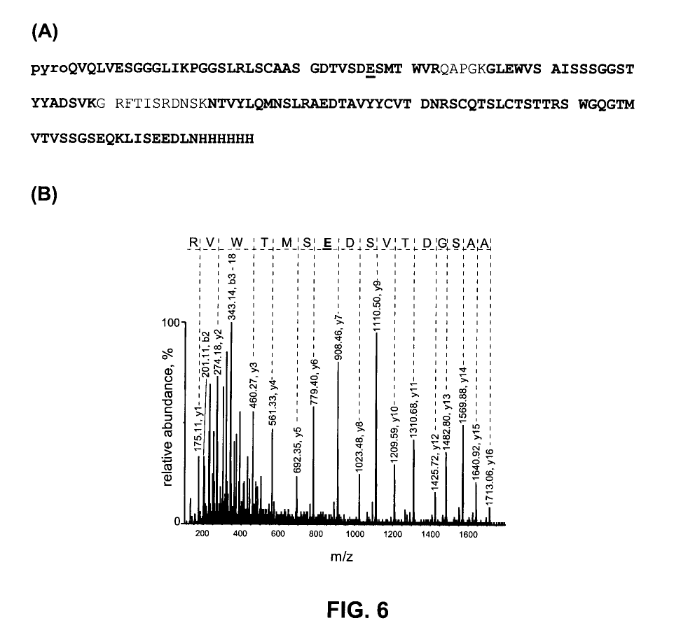

The identification coverage of huVHAm302 from the analysis of the tryptic protein digest using nanoRPLC-MS/MS with data dependent analysis (DDA) was 86% (FIG. 6A; SEQ ID NO:12). A prominent doubly protonated ion at m/z 1036.47 (2+) was sequenced as LSCamAASGDTVSDESMTWVR (SEQ ID NO:13; Cam is carboxyamidomethylated cysteine, whose residue mass is 160.03 Da) for residues 20-38 of huVHAm302 (FIG. 6B). Peptide ions from LSCamAASGDTVSDQSMTWVR (SEQ ID NO:14) were not detected at all indicating 100% occupancy of glutamic acid (underlined) at position 32 of huVHAm302. The remaining amino acid sequence was identical to that expected for huVHAm302. The possibility that the amber codon was read as Q during translation but later deaminated to E is excluded, as all the other Qs (see tryptic fragments in FIG. 6A) were indeed Q. Immediately following its His₆ tag, huVHAm302 had another amber codon preceding a TM translation stop codon. To provide a second example, the identity of the amino acid coded by this second amber codon was determined. However, the mass spectrometry results showed that in this case the amber codon was completely read as stop codon. The amber codons are known to be inefficiently suppressed in suppressor strains, e.g., TG1 E. coli, when they are followed by a T or C (Miller, J. H. et al., 1983) (Bossi, L., 1983). The determined molecular weight of the protein (15,541.2 Da) also confirmed that huVHAm302 had the His₆ tag as its last residues. Therefore, all the V_{H}s were recloned, substituting the amber codon with a Glu codon.

### Example 4

### Production of Soluble Human V_{H}s

The nine V_{H}s, huVHAm302 (SEQ ID NO:15), huVHAm304 (SEQ ID NO:16), huVHAm309 (SEQ ID NO:17), huVHAm315 (SEQ ID NO:18), huVHAm316 (SEQ ID NO:19), huVHAm416 (SEQ ID NO:20), huVHAm427 (SEQ ID NO:21), huVHAm428 (SEQ ID NO:22) and huVHAm431 (SEQ ID NO:23), were subcloned, substituting the amber codon with a Glu codon.

V_{H} genes were sub-cloned into pSJF2H vector for soluble expression in E. coli strain TG1 (Arbabi-Ghahroudi et al., 2009) using the primers HVHP430Bam and HVHP430Bbs. The V_{H} silent mutants with their amber codon at position 32 replaced with Glu codon were constructed by SOE and PCR using pSJF2H vectors containing V_{H} genes as templates (Ho et al., 1989) (Yau et al., 2005). In each case, specific mutagenic primers were included to amplify two fragments which had the aforementioned mutation in CDR1 gene. The two fragments were then spliced together by SOE, amplified again by PCR and cloned for expression. Expression and purification were carried out (Arbabi-Ghahroudi et al., 2009). Size exclusion chromatography of the purified V_{H}s was performed with a Superdex™ 75 column (GE Healthcare, Baie d'Urfé, QC, Canada).

Size exclusion chromatography of the V_{H}s showed a significant improvement in the solubility of V_{H}s. FIG. 5 shows graphs illustrating the aggregation tendencies of V_{H}s in terms of the percentage of their monomeric contents. Percent monomer was obtained by integrating the area under the monomeric and multimeric peaks from size exclusion chromatograms. “Mono” denotes V_{H}s identified by panning in monovalent phage display format. All the V_{H}s had basic pI (9.1±0.3, mean ±SD). “Multi/Ht” denotes V_{H}s identified by panning in multivalent display with a heat denaturation step. The median values, shown by horizontal bars are 78% for “Mono V_{H}s ” and 90% for “Multi/Ht V_{H}s.” The inset shows the aggregation states of Multi/Ht V_{H}s as a function of their pIs.

Compared to the V_{H}s isolated by panning in a monovalent display format (median: 78%), the V_{H}s isolated by heat denaturation in a multivalent display format show a higher proportion of monomer contents (median: 90%) with four (huVHAm304, huVHAm309, huVHAm416, huVHAm428) being completely monomeric (FIGS. 4B and 5). Interestingly, of these four V_{H}s, three are acidic (huVHAm304, pI 5.3; huVHAm416, pI 5.8; huVHAm428, pI 5.8), whereas only one was basic (huVHAm309, pI 8.2) (FIG. 5 inset; Table 2). The remaining five V_{H}s were basic or almost neutral (Table 2). Of the four V_{H}s with the least monomeric contents three (huVHAm315, huVHAm427, huVHAm302) had pIs around the neutral pH (7.3, 7.0, 6.4).

Previously it was observed that a V_{H} obtained with the heat denaturation approach had an acidic pI (5.7) and showed a reversible folding upon heat denaturation; however, other V_{H}s obtained without the heat step had higher pIs (7.4±1.2, mean ±SD) and did not show reversible heat denaturation (Jespers et al., 2004a). Also of the six aggregation-resistant protein A binding V_{H}s, four had acidic pI (4.3-4.7) whereas two, C85 and C36, had neutral (7.0) and basic (8.0) pIs, respectively. Also, a highly refoldable and non-aggregating lysozyme-specific V_{H}, HEL4 (Jespers et al., 2004b), was also shown to have a very acidic pI, 4.7. All the aggregating V_{H}s isolated by the method of the present invention with the monovalent display format had basic pIs (9.1±0.3, mean ±SD) (FIG. 5). Interestingly, the analysis described in Example 8 (see below) show that all the non-aggregating, acidic V_{H}S (Jespers et al., 2004b; Jespers et al., 2004a) have pIs less than 6.

### Example 5

### Alkylation Reactions and Molecular Mass Determinations by Mass Spectrometry

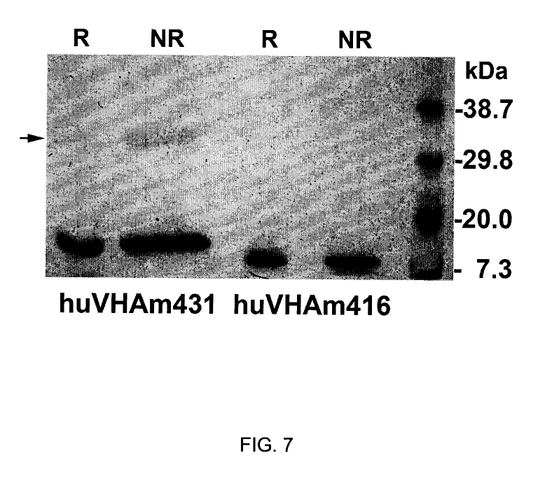

SDS-PAGE analyses of the five aggregating V_{H}s (huVHAm302, huVHAm315, huVHAm316, huVHAm427 and huVHAm431) revealed dimer species on non-reducing gels but not on reducing gels for four of the V_{H}s, indicating the existence of inter-domain disulfide linkages in these V_{H}s (FIG. 7; Table 1). Thus, for these V_{H}s the non-canonical Cys residues contribute to their aggregation. The four non-aggregating V_{H}s were further tested for the presence of intra- and inter-CDR disulfide linkages by alkylation reaction/mass spectrometry experiments.

Alkylation reactions/mass spectrometry was conducted according to Tanha et al. (2001) with iodoacetamide as the alkylating reagent. Briefly, Cold acetone (5×vol) was added to 30 μg of VH solution and the contents were mixed and centrifuged in a microfuge at maximum speed at 4° C. for 10 min. The pellet was exposed to air for 5 min, dissolved in 250 μL of 6 M guanidine hydrochloride and 27.5 μL of 1 M Tris buffer, pH 8.0, was added. 20×DTT in molar excess of Cys residues was added and the mixture was incubated at room temperature for 30 min. A 5 molar excess, relative to DTT, of freshly-made iodoacetamide was added and the reaction was incubated at room temperature for 1 h in the dark. The alkylated product was dialyzed in 3.5 L of ddH₂O at 4° C. using a Slide-A-Lyzer™ with 10 kDa MWCO (Pierce, Rockford, Ill.). The reaction solutions were reduced to 15 μL with a SpeedVac and were subsequently subjected to MALDI mass spectrometry for molecular mass determination of V_{H}s. Control experiments were identical except that DTT was replaced with ddH₂O.

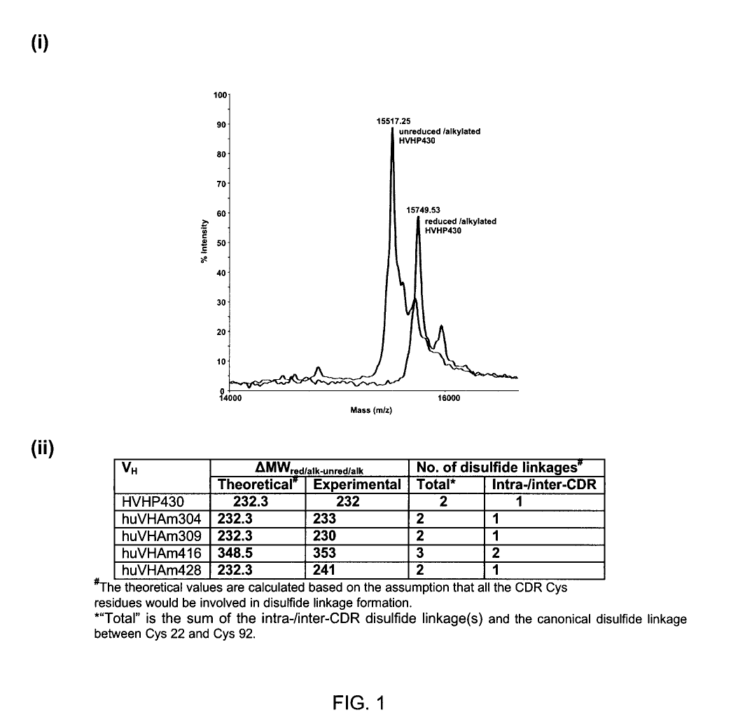

FIG. 1 illustrates (i) molecular mass profiles obtained by mass spectrometry of unreduced/alkylated (unred/alk) and reduced/alkylated (red/alk) HVHP430 V_{H}. FIG. 1(ii) presents the results of alkylation reaction/mass spectrometry experiments for HVHP430 and four anti-α-amylase V_{H}s identified in this study. All the V_{H}s have c-Myc-His₅₍₆₎ tags. The mass spectrometry profiles of the HVHP430 V_{H}s are combined to provide a better visual comparison. The unreduced, iodoacetamide-treated V_{H} has a mass of 15,517.25 Da, a mass expected for an unalkylated V_{H} (15,524.39 Da). In contrast, the reduced, iodoacetamide-treated V_{H} shows a mass increase of 232.32 Da with respect to the unreduced V_{H}, indicating alkylation at all four Cys residues (4×58.08 Da=232. 32 Da). The observation that V_{H} alkylation occurs only after reducing the Cys sulfhydride groups demonstrates that the two CDR3 Cys residues are engaged in an intra-CDR3 disulfide linkage.

As shown in FIG. 1(ii) all the CDR cysteines are engaged in disulfide linkages. Thus, huVHAm304 and huVHAm309 have intra-CDR3 disulfide linkages, huVHAm428 has a CDR1-CDR3 disulfide linkage and huVHAm416 has both intra- and inter-CDR disulfide linkages.

### Example 6

### Thermal Refolding Efficiency Experiments

The four non-aggregating V_{H}s (huVHAm304, huVHAm309, huVHAm416 and huVHAm428) were examined for their reversible thermal unfolding status by comparing the KDs for the binding of the native (KDn) and heat-treated/cooled (KDref) V_{H}s to protein A (To et al., 2005).

Thermal refolding efficiency of V_{H}s at concentrations of 0.5 and 5 μM was determined by measuring the binding of native and heat denatured/cooled V_{H}s to protein A from surface plasmon resonance (SPR) data collected with BIACORE 3000 biosensor system (Biacore Inc., Piscataway, N.J.). 600 resonance units (RUs) of protein A (Sigma) or ovalbumin (Sigma) as a reference protein were immobilized on research grade CM5-sensorchip (Biacore Inc.). Immobilizations were carried out at a protein concentration of 50 μg/mL in 10 mM acetate buffer pH 4.5 using amine coupling kit supplied by the manufacturer. AD V_{H}s were passed though Superdex™ 75 column (GE Healthcare) and the monomeric species were collected for refolding efficiency experiments. To obtain refolding efficiency values, V_{H}s were incubated at 85° C. for 20 min at the concentration of 0.5 and 5 μM and were cooled to room temperature for 30 min. The V_{H}s were centrifuged at 16,000 g in a microfuge for 5 min at 22° C. to pellet and remove any possible aggregates. Binding analyses of native and heat denatured/cooled V_{H}s against protein A were carried out at 25° C. in 10 mM HEPES, pH 7.4 containing 150 mM NaCl, 3 mM EDTA and 0.005% surfactant P20 at a flow rate of 40 μL/min. The surfaces were washed thoroughly with the running buffer for regeneration. Refolding efficiencies were calculated from the amounts bound at steady state. Data were analyzed with BIAevaluation 4.1 software.

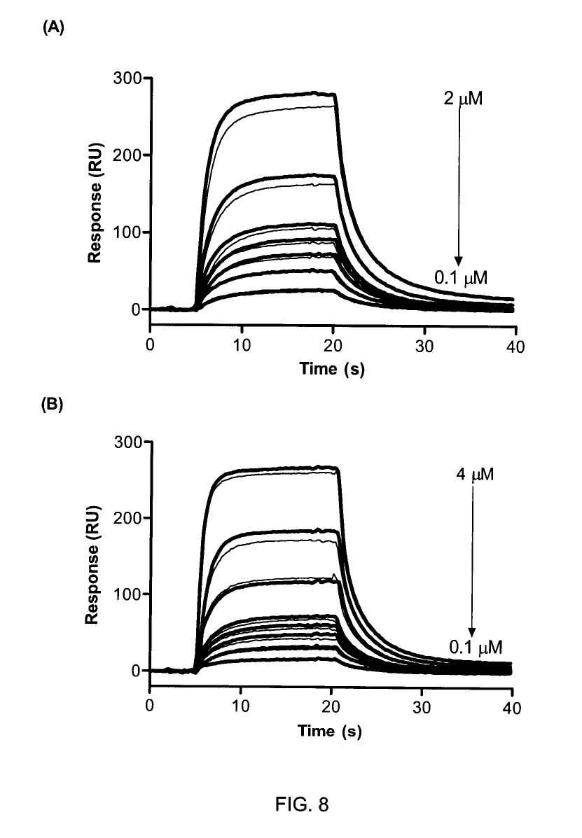

FIG. 8 shows sensorgram overlays showing the binding of native (thick lines) and refolded (thin lines) huVHAm309 (A) and huVHAm416 (B) to immobilized protein A at 0.1, 0.2, 0.3, 0.4, 0.5, 1 and 2 μM (huVHAm309) and 0.1, 0.2, 0.3, 0.4, 0.5, 1, 2 and 4 μM (huVHAm416). KDn and KDref were calculated from respective sensorgrams and used to determine TREs. Data are for thermal unfolding of V_{H}s at 5 μM concentrations (KDn, KD of the native V_{H}; KDref, KD of the refolded (heat denatured/cooled) V_{H}).

The ratio of KDn to KDref defined as thermal refolding efficiency (TRE) gives a measure of the degree the V_{H}s refold to their native state following thermal denaturation. The denaturation and measurement of TREs were performed at two different V_{H} concentrations: 0.5 and 5 μM (FIG. 8; Table 2). Only huVHAm304 showed a concentration-dependent TRE where its TRE decreased from 99% at 0.5 μM to 87% at 5 μM. Aggregation formation which is accelerated at higher protein concentrations is most likely the cause of this decrease. However, for all V_{H}s, the TRE values were still very high at 5 μM ranging from 86% to 97%. The highest TRE is demonstrated by huVHAm416 which has one more non-canonical disulfide linkage than the other three (see above), underlining the importance of non-canonical disulfide linkages in single domain stability.

### Example 7

### α-amylase Binding and Inhibition Assays

The four monomeric V_{H}s, huVHAm304, huVHAm309, huVHAm416 and huVHAm428 were chosen for further binding analysis against α-amylase

α-amylase inhibition assays were performed essentially as described (Lauwereys, M. et al., 1998). Briefly, the enzyme at a final concentration of 1.5 μg/mL in 0.1% casein, 150 mM NaCl, 2 mM CaCl₂, 50 mM Tris-HCl pH 7.4 was preincubated with various concentrations of purified monomeric anti-α-amylase V_{H}s at room temperature for 1 h (total volume: 50 μL). The mixture was split in two ELISA wells and to each well 75 μL of substrate solution (0.2 mM 2-chloro-4-nitrophenyl maltotrioside, 150 mM NaCl, 2 mM CaCl₂, 50 mM Tris-HCl pH 7.4) was added. Controls reactions included ones with no V_{H} and ones with HVHP430 V_{H} at all V_{H} concentrations tested. The progress of reactions was monitored continuously at 25° C. by measuring the change in absorbance of reaction solutions at 405 nm (ΔA405 nm) using a PowerWave 340 microplate spectrophotometer (BioTek Instruments, Inc. Winooski, Vt.). Enzyme's residual activity was calculated relative to its activity in the presence of the non-binder, library scaffold, HVHP430 V_{H}. Equilibrium dissociation constants by Biacore could not be determined, because α-amylase lost its activity upon immobilization on Biacore chips. The V_{H}s was thus analyzed by ELISA.

To assess binding of V_{H}s to a-amylase by ELISA, Maxisorp™ microtiter plates (Nunc) were coated with 100 μL of 10 μg/mL porcine pancreatic α-amylase (Sigma, Oakville, ON, Canada) in PBS overnight at 4° C. After blocking with 3% bovine serum albumin (300 μL) for 2 h at 37° C. and subsequent removal of blocking agent, 100 μL His₆-tagged V_{H}s at concentrations of a few μM were added, followed by incubation for 2 h at 37° C. Wells were washed 5× with PBST and 100 μL rabbit anti-His-IgG/horse radish peroxidase (HRP) conjugate (Bethyl Laboratories, Inc., Montgomery, Tex.) was added at a dilution of 1:5000. The wells were then incubated for 1 h at 37° C. After washing the wells with PBST, 100 μL ABTS substrate (KPL, Gaithersburg, Md.) was added and the reaction, seen as color development, was stopped after 5 min by adding 100 μL of 1 M phosphoric acid. Absorbance values were measured at a wavelength of 450 nm using a microtiter plate reader. The protein A binding activity of the V_{H}s was assessed as above where a protein A/HRP conjugate (Upstate, Lake Placid, N.Y.) was added as the detection reagent to the wells coated with V_{H}s. Assays were performed in duplicates.

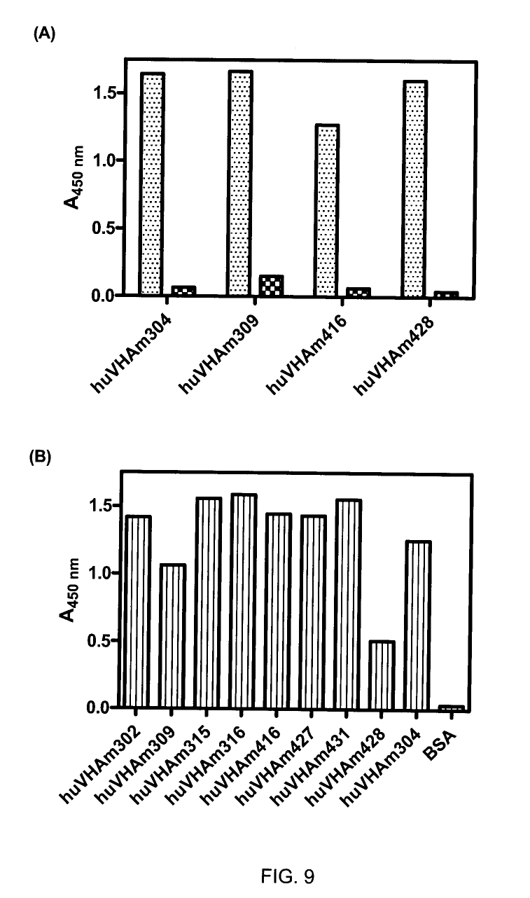

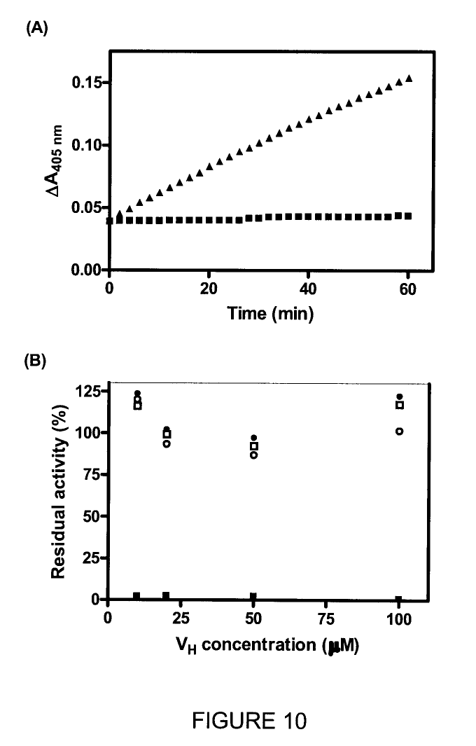

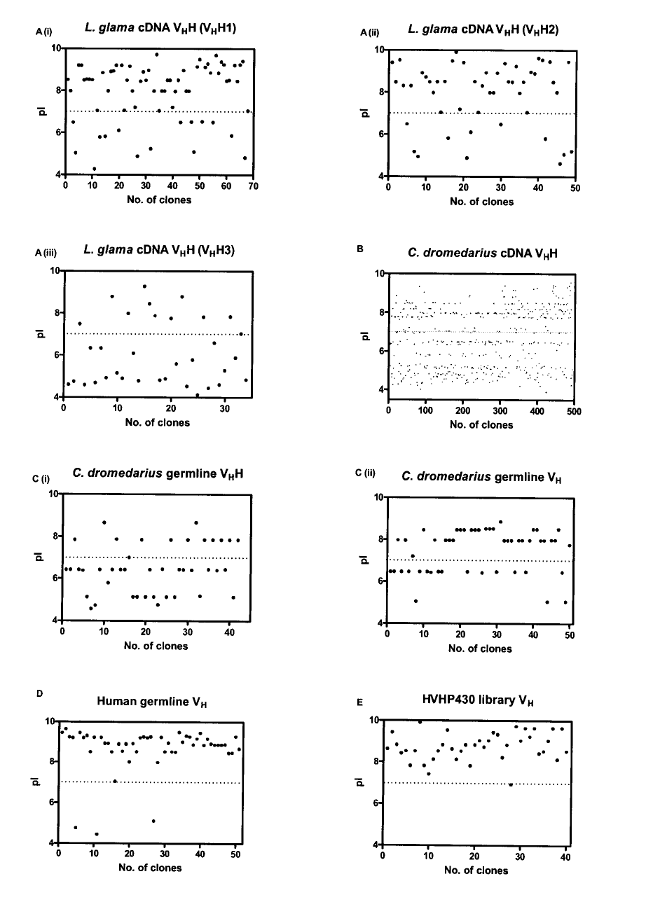

As shown in FIG. 9A, all four clones bound to α-amylase. They, as well as the other five V_{H}s (huVHAm302, huVHAm315, huVHAm316, huVHAm427 and huVHAm431), bound to protein A (Table 1, FIG. 9B). Moreover, of the four V_{H}s tested in enzyme inhibition assays, one (huVHAm302) which also formed intra-CDR3 disulfide linkage (see Table 1) inhibited α-amylase (FIG. 10).

### Example 8

### Analysis of the Isoelectric Points of V_{H} and V_{H}H Domains

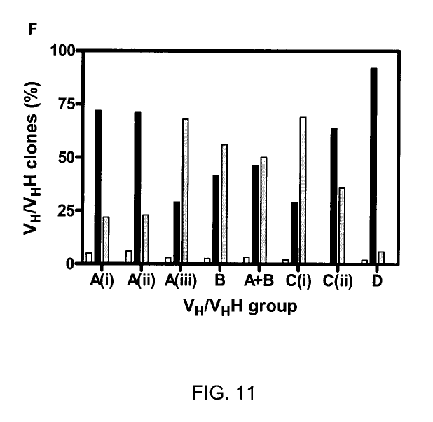

A theoretical pI distribution analysis was conducted of Lama glama cDNA V_{H}Hs (Harmsen et al., 2000; Tanha et al., 2002), Camelus dromedarius cDNA V_{H}Hs (NCBI, Accession Nos. AB091838-AB092333), C. dromedarius germline V_{H}H and V_{H} segments (Nguyen et al., 2000) and human germline V_{H} segments (V BASE; http://vbase.mrc-cpe.cam.ac.uk/) using Laser gene V6.0 software. FIGS. 11A-F show graphs illustrating theoretical pI distribution (A-F) for L. glama cDNA V_{H}Hs of subfamilies V_{H}H1, V_{H}H2 and V_{H}H3, C. dromedarius cDNA V_{H}Hs, germline V_{H}H segments and germline V_{H} segments, human germline V_{H} segments and the HVHP430 library V_{H}s. The dotted line denotes pI 7.0. In F, for each of the seven V_{H}/V_{H}H group (A-D), percentage of the clones with neutral pI (white bars), basic pI (black bars) and acidic pI (grey bars) are shown. A+B shows the composite profile obtained by pooling the L. glama and C. dromedaries cDNA V_{H}Hs together.

Regarding L. glama cDNA V_{H}Hs from V_{H}H1 subfamily (68 clones), 22% of the V_{H}Hs are acidic compared to 72% basic. The figures for V_{H}H2 subfamily members (49 clones) are comparable: 23% acidic versus 71% basic. Conversely, for V_{H}H3 subfamily (34 clones), 68% of the V_{H}Hs have acidic pl, versus 29% with basic pI. However, many of the sequence entries do not have the first few FR1 amino acids, which often have acidic amino acids at position 1. With an acidic residue included in FR1, the proportion of the acidic V_{H}Hs could be as high as 34% (V_{H}H1), 37% (V_{H}H2) and 79% (V_{H}H3). C. dromedaries V_{H}H pool (495 clones [NCBI, Accession Nos. AB091838-AB092333]) shows a similar pattern to the L. glama one of V_{H}H3 subfamily, consisting mostly of acidic V_{H}Hs (56% acidic versus 41% basic). Interestingly, of the three L. glama V_{H}H subfamilies, V_{H}H3 subfamily is also the one with which C. dromedarius V_{H}Hs shares structural features the most. The composite figure, taking into consideration all 646 camelid V_{H}Hs, for acidic V_{H}Hs is 50% which can be as high as 53% with the inclusion of the acidic residue at position 1 (versus 43% for basic V_{H}Hs). A comparison of C. dromedarius germline V_{H} segments versus V_{H}H segments reveals that while for V_{H}s, the pI distribution pattern is 64% basic versus 36% acidic, for V_{H}Hs the pattern is reverse: 69% acidic versus 29% basic. In the instance of human germline V_{H} segments, the overwhelming majority of V_{H}s have basic pI: 92% basic versus 6% acidic (1-f V_{H} segment, pI 4.4; 1-24 V_{H} segment, pI 4.7; 3-43 V_{H} segment, pI 5.1). Of the 36 library clones analyzed, none had acidic pI (8.7 ±0.7, mean ±SD) (FIG. 11E). Thus, based on the biophysical and statistical date accumulated so far on human and camelid V_{H}s/V_{H}Hs in this study and previously (Jespers, L. et al., 2004b; Jespers, L. et al., 2004a) it is possible that the high abundance of acidic V_{H}Hs in camelid sdAb repertoire is not a random occurrence, rather the result of nature arriving at a solution to generate soluble and stable sdAbs by in vivo evolution. Protein acidification may be another approach to creating functional single domains.

### Example 9

### Cloning Llama V_{H}H CDR3 Repertoire

A plasmid library of llama V_{H}H CDR3s was constructed in E. coli. Two hundred and sixty nanogram of RNA, purified from 110 μL of a llama (Lama glama) blood by QIAamp RNA Blood Mini™ kit (QIAGEN Inc.), was used as template to synthesize cDNA using the First-Strand cDNA Synthesis™ kit (GE Healthcare) and pd(T)18 provided by the manufacturer. The entire cDNA prep was amplified by PCR using the primer pairs VHHFR3Bgl-R/CH2B3-F, VHBACKA6/CH2B3-F, VHHFR3Bgl-R/CH2FORTA4 and VHBACKA6/CH2FORTA4 (see Table 3 for a list of primers and subsection ‘HVHP430LGH3 V_{H} Library Construction’). The amplified products were run on agarose gels and the bands derived from heavy-chain antibodies were gel-purified using QIAquick Gel Extraction™ kit (QIAGEN Inc.). A total of 730 ng of purified DNA was subjected to a second round of PCR using the primer pair VHHFR3Bgl-R/VHHFR4Bgl-F. The amplified products were digested with Bgl II and purified by QIAquick PCR Purification™ kit (QIAGEN Inc.). Examination of the 174 V_{H}H sequences had shown that only two V_{H}Hs had internal Bgl II restriction sites in their CDR3). The cloning vector, pSJF2, (Tanha et al., 2003) was digested with Bgl II and gel-purified. Ligation reaction was performed at 16° C. overnight in a total volume of 200 μL and contained 1.25 μg of total digested DNA at 2:1 insert:vector molar ratio and 4 μL 400 units/μL DNA ligase (NEB, Pickering, ON, Canada) in the buffer provided by the manufacturer. The ligation product was desalted using the PCR purification kit and eluted with 90 μL deionized water. To transform, 50 μL of E. coli TG1 cells were mixed with 5 μL of the ligation product and electroporated (Tanha et al., 2001). Following transformation, cells were transferred immediately to 1 mL SOC medium (Sambrook et al., 1989). A total of 18 electroporations were performed. The electroporated cells were pooled (total volume=18 mL) and incubated at 37° C. for 1 h at 220 rpm. Small aliquots were removed, and serial dilutions of the cells in LB medium were made and spread on LB plates containing 100 μg/mL ampicillin and the titer plates were incubated at 32° C. overnight. To the remaining cells in SOC, ampicillin was added to a final concentration of 100 μg/mL followed by incubation at 37° C. for 2.5 h at 220 rpm. The culture was transferred to a flask containing 1 L of LB plus 100 μg/mL ampicillin and incubated at 37° C. overnight at 220 rpm. 100 mL was used to obtain a stock of purified library plasmid using Plasmid Maxi™ kit (QIAGEN Inc.), the remainder was centrifuged and the pelleted cells were resuspended in 15% glycerol in LB and stored frozen in small aliquots at −80° C. The number of colonies on the titer plates was used to calculate the size of the library. CDR3 sequences were amplified by colony PCR of single colonies from the titer plates, purified (QIAquick PCR Purification™ kit) and sequenced.

The plasmid library of llama V_{H}H CDR3s had 9.3×10⁸ independent transformants. Ninety one V_{H}H clones were selected from the library titer plates and sequenced. All had legitimate CDR3 sequences ranging in length from 5 to 31 amino acids with a mean/median value of 15 amino acids (FIG. 12). Fifteen CDR3 sequences were present more than once (2-5 times) (FIG. 12A; SEQ ID NOs:24-90). The inventors encountered such repetition of V_{H}H clones with identical CDR3 in previous studies (data not shown). Also, others, in a sample of about 170 rearranged L. glama V_{H}Hs, found several V_{H}Hs with at least 80% sequence identity in CDR3 (Harmsen et al., 2000). Eighteen clones (13 different sequences) had Cys residues in CDR3, predominantly the ones with longer CDR3 as observed before (Harmsen et al., 2000). Four clones had two Cys residues (Harmsen et al., 2000). Ten CDR3 sequences could be traced back to their V_{H}H2 subfamily origin since they had Asn or His at position 93 (Harmsen et al., 2000). As for the origin of the remaining CDR3, a definitive conclusion cannot be drawn but it is very likely that at least some of the CDR3s with Cys are derived from V_{H}Hs from V_{H}H3 subfamily. Additionally, it is possible that many of the shorter CDR3s are of V_{H}H1 and V_{H}H2 subfamily origin, while the longer ones are derived from V_{H}H3 family.

### Example 10

### HVHP430LGH3 V_{H} Library Construction

A V_{H} synthetic phage display library based on HVHP430 V_{H} scaffold was constructed. The diversity of the library was generated by surmounting the CDR3 sequences from the V_{H}H CDR3 plasmid library and the H1/CDR1 sequences from the HVHP430 phage display library as described herein. Generating diversity by in vitro CDR randomization may also result in V_{H} species in the library that are insoluble. V_{H}H CDR3s, however, are known to solubilize V_{H}Hs (Desmyter et al., 2002) (Vranken et al., 2002) (Tanha et al., 2002 and references therein) and may have been evolutionarily selected for this purpose. It was for their solubilization property that llama V_{H}H CDR3 was incorporated into the inventors' library to minimize the proportion of insoluble V_{H}s, while at the same time creating diversity.

Primers used for library construction are listed in Table 3 below; the first two primers are already in Table 2. The FR3- and FR4-specific primers, VHHFR3Bgl-R and VHHFR4Bgl-F, were designed based on alignment of nucleotide sequences of 174 L. glama V_{H}Hs belonging to subfamilies V_{H}H1, V_{H}H2 and V_{H}H3 (Harmsen et al., 2000;Tanha et al., 2002).

TABLE 3
Primers used to construct HVHP430LGH3 V_{H} phage display
library

| designation | sequence |
|---|---|
| HVHBR1-R | 5′- |
| (SEQ ID NO: 91) | CATGTGTAGACTCGCGGCCCAGCCGGCCATGGCCCAGGTGCAGC |
| | TGGTGGAGTC-3′ |
| HVHFR4-F | 5′- |
| (SEQ ID NO: 92) | CATGTGTAGATTCCTGGCCGGCCTGGCCTGAAGAGACGGTGACC |
| | ATTGTCC-3′ |
| VHHFR3Bgl-R | 5′-ACTGACAGATCTGAGGACACGGCCGTTTATTACTGT-3′ |
| (SEQ ID NO: 93) | |
| VHHFR4Bgl-F | 5′-ACTGACAGATCTTGAGGAGACGGTGACCTG-3′ |
| (SEQ ID NO: 94) | |
| VHBACKA6* | 5′-GATGTGCAGCTGCAGGCGTCTGGRGGAGG-3′ |
| (SEQ ID NO: 95) | |
| CH2B3-F | 5′-GGGGTACCTGTCATCCACGGACCAGCTGA-3′ |
| (SEQ ID NO: 96) | |
| CH2FORTA4* | 5′-CGCCATCAAGGTACCAGTTGA-3′ |
| (SEQ ID NO: 97) | |
| P430FR3-R | 5′-CTGAGGACACGGCTGTGTATTACTGT-3′ |
| (SEQ ID NO: 98) | |
| P430FR3-F | 5′-ACAGTAATACACAGCCGTGTCCTCAG-3′ |
| (SEQ ID NO: 99) | |
| P430FR4Mod-F | 5′-TGAGGAGACGGTGACCATGGTCCCCTGGCCCCA-3′ |
| (SEQ ID NO: 100) | |

To construct the library, two overlapping fragments were generated by standard PCRs. The first, upstream fragment containing a randomized H1/CDR1 was generated using the HVHP430 library phagemids as the template and primers HVHBR1-R and P430FR3-F. The second, downstream fragment containing the llama V_{H}H CDR3 repertoire was generated using the V_{H}H CDR3 repertoire plasmids as the template and primers P430FR3-R and P430FR4Mod-F. The two fragments were gel-purified (Qiagen Inc.), mixed in equimolar amount and spliced/amplified by splice overlap extension/PCR to construct full length V_{H} genes. SOE/PCR was carried out using Expand high fidelity DNA polymerase system (Hoffmann-La Roche Limited, Mississauga, ON, Canada) and framework 1- and 4-specific primers HVHBR1-R and HVHFR4-F, respectively, which were tailed with non-compatible Sfi I restriction enzymes sites. The V_{H} fragments were purified with QIAquick PCR Purification™ kit (Qiagen Inc.), digested along with the phagemid vector (pMED1) overnight with Sfi I enzyme. To minimize vector self ligation during ligation reactions, pMED1 was further digested for 3 h with Pst I and Xho I which have recognition sites between the two Sfi I sites. The digested vector and V_{H} preparations were subsequently purified by the PCR purification kit and were ligated in a 1:2 molar ratio, respectively, using LigaFast™ Rapid DNA Ligation System (Promega, Madison, Wis.). A total of 112.5 μg vector and 20 μg V_{H} were combined, ligation buffer and T4 DNA ligase were added and the contents were mixed and incubated for 2 h at room temperature. The ligated materials were subsequently purified by the PCR purification kit and concentrated to approximately 1 μg/μL. Transformations were performed by a standard electroporation using a mixture of 50 μL of electrocompetent TG1 cells (Stratagene, La Jolla, Calif.) and 2 μL of ligated material per electroporation cuvette. A total of 50 electroporations were performed. After each electroporation, the electroporated bacterial cells were diluted in 1 mL SOC medium and incubated in a shaker incubator for 1 h at 37° C. and 200 rpm. Following incubation, an aliquot was removed for library size determination purposes, and the remaining cell library was amplified in 200 mL of 2×YT containing 100 μg/mL ampicillin and 2% glucose (2×YT/Amp/2% Glu) overnight at 37° C. and 200 rpm. The cells were pelleted by centrifugation, resuspended in a final volume of 20 mL of 35% glycerol in YT/Amp/1% Glu and stored in one-mL aliquots at −80° C.

The size of the HVHP430LGH3 phage display library was 4.5×10⁸. Thirty one clones from the library were selected at random and their V_{H}s were sequenced as set out in Table 4.

TABLE 4
Sequence of CDR3 for 31 clones from the HVHP430LGH3
V_{H} phage display library.

| Clone | H1/CDR1 | SEQ ID NO. | 93-102 (93/94/CDR3) | SEQ ID NO. |
|---|---|---|---|---|
| HLlib25 | FMFSN*IMS | 101 | AVDEGLLYNDNYYFTLHPSAYDY | 132 |
| HLlibM6 | DSVTHECMT | 102 | GQGQGLYNSVADYYTGRADFDS | 133 |
| HLlib12 | VRFIDEVMG | 103 | ITVQLNPVVFGAGWIIDYNY | 134 |
| HLlibM14 | FNFIAETMT | 104 | AAATRPSIAFPISVGAYET | 135 |
| HLlibM5 | VMLNHECMT | 105 | VTLYDAVCATYVPEGLRDY | 136 |
| HLlibM3 | YILTAESMT | 106 | VTNTNYLSF*RASIVRSF | 137 |
| HLlib18 | FIFSYEGMG | 107 | AANQGGHSRFAQRYDY | 138 |
| HLlibM16 | TIIIPECMT | 108 | TLTQAC*TACRIGPPS | 139 |
| HLlibM18 | FNFSAEIMT | 109 | PNWSRLTHQCSPNMSY | 140 |
| HLlib16 | VSFSA*FMA | 110 | GARIGWYTCRYDYDY | 141 |
| HLlibM12 | DNFTPEFMS | 111 | GARIGWYTCRYDYDY | 142 |
| HLlib05 | VMFTP*DMG | 112 | YLQLFRSTTRSYDTY | 143 |
| HLlibM9 | FTSIAEVMG | 113 | AADIRSPSRFSISGY | 144 |
| HLlibM7 | VKFTSKSMT | 114 | VGITMSVVG*LCARY | 145 |
| HLlibM15 | TNLTHETMA | 115 | AAGPTLSTDAYEYRY | 146 |
| HLlib02 | FNISTYFMG | 116 | NADYFRGNSYRTMT | 147 |
| HLlib10 | YMVIS*AMA | 117 | NARQWKNTDWVDY | 148 |
| HLlib13 | YMFSYEVMG | 118 | NARQWKNTDWVDY | 149 |
| HLlibM1 | YSVTTETMS | 119 | NARQWKNTDWVDY | 150 |
| HLlibM5 | FMFTPETMA | 120 | NARQWKNTDWVDY | 151 |
| HLlib14 | FIVNDESMT | 121 | AAKKIDGPRYDY | 152 |
| HLlib23 | YTLSYEIMA | 122 | NARTGSGLREY | 153 |
| HLlib21 | FMLSSYAMT | 123 | NAMKRLYCMTT | 154 |
| HLlib28 | VRFSDEFMG | 124 | YARSVRSPDDY | 155 |
| HLlibM17 | DIFIAESMG | 125 | VTTMNPVPAPS | 156 |
| HLlibM8 | DMFSHESMG | 126 | NAESSAVPYDY | 157 |
| HLlibM9 | DSLSYENMT | 127 | TVRGPYGSSRY | 158 |
| HLlib01 | FMFSS*CMA | 128 | TTSPFGTPNY | 159 |
| HLlib15 | FKFSYECMG | 129 | AADLLSGRL | 160 |
| HLlib19 | FTLNTEFMA | 130 | NAQNW | 161 |
| HLlib1 | YSFNSESMG | 131 | VAWF | 162 |

*coded by amber stop codon, overwritten as Glu

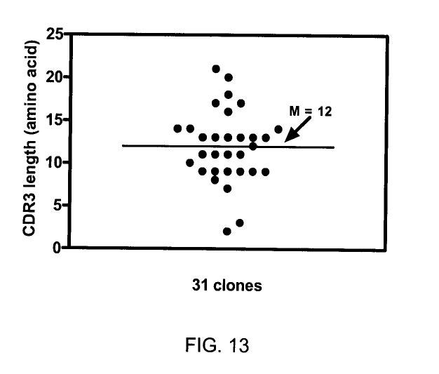

The V_{H}s were different with respect to H1/CDR1, but six showed sequence overlap with respect to CDR3 (HLlib16 and HLlibM12; HLlib10, HLlib13, HLlibM1 and HLlibM5). In fact, the latter four clones had the same CDR3 as clone CH2-16A from the plasmid CDR3 library. Interestingly, 28 out of the 31 clones had the acidic residue E at position 32. The lengths of CDR3s ranged from 2-21 amino acids with a mean/median value of 12 (Table 4 and FIG. 13).

### Example 11

### Production of Library Phages

A 1-mL frozen aliquot of the library (˜5×10¹⁰ cells) was thawed on ice, mixed with 200 mL 2×YT/Amp/1% Glu and grown at 37° C. and 220 rpm to an OD₆₀₀ of 0.5. The culture was infected with helper phage at 20:1 ratio of phage to bacterial cells and incubated for 15 min without shaking followed by 1 h incubation at 37° C. with shaking at 200 rpm. Bacterial cells were then pelleted by centrifuging at 3,000 g for 10 min and resuspended in 200 mL of 2×YT/Amp containing 50 μg/mL kanamycin. The culture was incubated in a shaker incubator overnight at 37° C. and 220 rpm. Phages were purified in a final volume of 2 mL sterile PBS, aliquoted and stored frozen at −20° C. Phage titrations were performed as described (Arbabi et al., 2009).

Panning is performed as previously described.

The embodiments and examples described herein are illustrative and are not meant to limit the scope of the invention as claimed. Variations of the foregoing embodiments, including alternatives, modifications and equivalents, are intended by the inventors to be encompassed by the claims. Furthermore, the discussed combination of features might not be necessary for the inventive solution.

### REFEERNCES

All patents, patent applications and publications referred to herein are hereby incorporated by reference.
Aiyar, A., Xiang, Y., and Leis, J. (1996). Site-directed mutagenesis using overlap extension PCR. Methods Mol. Biol. 57: 177-191.
Arbabi-Ghahroudi, M., Desmyter, A., Wyns, L., Hamers, R., and Muyldermans, S. (1997). Selection and identification of single domain antibody fragments from camel heavy-chain antibodies. FEBS Lett 414: 521-526.
Arbabi-Ghahroudi, M., Tanha, J., and MacKenzie, R. (2009) Isolation of monoclonal antibody fragments from phage display libraries. Methods Mol. Biol. 502: 341-364
Arbabi-Ghahroudi, M., To, R., Gaudette, N., Hirama, T., Ding, W., MacKenzie, R., and Tanha, J. (2008). Aggregation-resistant VHs selected by in vitro evolution tend to have disulfide-bonded loops and acidic isoelectric points. Protein Eng Des Sel. Electronic
Baca, M., Presta, L. G., O'Connor, S. J., and Wells, J. A. (1997). Antibody humanization using monovalent phage display. J.Biol.Chem. 272: 10678-10684.
Baek, H., Suk, K. H., Kim, Y. H., and Cha, S. (2002). An improved helper phage system for efficient isolation of specific antibody molecules in phage display. Nucleic Acids Res. 30: e18.
Bagshawe, K. D, (1987), Br. J. Cancer 56, 531-532.
Bagshawe, K. D. (2006). Antibody-directed enzyme prodrug therapy (ADEPT) for Cancer. Expert Review of Anticancer Therapy.Vol. 6, 10, Pages 1421-1431
Becerril, B., Poul, M. A., and Marks, J. D. (1999). Toward selection of internalizing antibodies from phage libraries. Biochem.Biophys.Res.Commun. 255: 386-393.
Bloom, J. D., Labthavikul, S. T., Otey, C. R., and Arnold, F. H. (2006). Protein stability promotes evolvability. Proc.Natl.Acad.Sci.U.S.A 103: 5869-5874.
Bond, C. J., Marsters, J. C., and Sidhu, S. S. (2003). Contributions of CDR3 to VHH domain stability and the design of monobody scaffolds for naive antibody libraries. J.Mol.Biol. 332: 643-655.
Bradbury, A. R. & Marks, J. D. (2004) Antibodies from phage antibody librairies. J. Immunol Methods 290, 29-49.
Christ, D., Famm, K., and Winter, G. (2006) Nucleic Acids Res. 34, e108
Christ, D., Famm, K., and Winter, G. (2007) Protein Eng. Des. Sel. 20, 413-416
Conrath, K. E., Lauwereys, M., Galleni, M., Matagne, A., Frere, J. M., Kinne, J., Wyns, L., and Muyldermans, S. (2001). Beta-lactamase inhibitors derived from single-domain antibody fragments elicited in the camelidae. Antimicrob.Agents Chemother. 45: 2807-2812.
Davies, J. and Riechmann, L. (1994). ‘Camelising’ human antibody fragments: NMR studies on VH domains. FEBS Lett 339: 285-290.
Davies, J. and Riechmann, L. (1995). Antibody VH domains as small recognition units. Biotechnology 13, 475-479.
Davies, J. and Riechmann, L. (1996). Single antibody domains as small recognition units: design and in vitro antigen selection of camelized, human VH domains with improved protein stability. Protein Eng 9: 531-537.
De, G. E., Silence, K., Decanniere, K., Conrath, K., Loris, R., Kinne, J., Muyldermans, S., and Wyns, L. (2006). Molecular basis for the preferential cleft recognition by dromedary heavy-chain antibodies. Proc.Natl.Acad.Sci.U.S.A 103: 4586-4591.
Decanniere, K., Desmyter, A., Lauwereys, M., Arbabi-Ghahroudi, M., Muyldermans, S., and Wyns, L. (1999). A single-domain antibody fragment in complex with RNase A: non-canonical loop structures and nanomolar affinity using two CDR loops. Structure. 7: 361-370.
Desmyter, A., Decanniere, K., Muyldermans, S., and Wyns, L. (2001). Antigen specificity and high affinity binding provided by one single loop of a camel single-domain antibody. J.Biol.Chem. 276: 26285-26290.
Desmyter, A., Spinelli, S., Payan, F., Lauwereys, M., Wyns, L., Muyldermans, S., and Cambillau, C. (2002). Three camelid VHH domains in complex with porcine pancreatic alpha-amylase. Inhibition and versatility of binding topology. J.Biol.Chem. 277: 23645-23650.
Desmyter, A., Transue, T. R., Arbabi-Ghahroudi, M., Thi, M. H., Poortmans, F., Hamers, R., Muyldermans, S., and Wyns, L. (1996). Crystal structure of a camel single-domain VH antibody fragment in complex with lysozyme. Nat.Struct.Biol. 3: 803-811.
Diaz, M., Stanfield, R. L., Greenberg, A. S., and Flajnik, M. F. (2002). Structural analysis, selection, and ontogeny of the shark new antigen receptor (IgNAR): identification of a new locus preferentially expressed in early development. Immunogenetics 54: 501-512.
Dooley, H., Flajnik, M. F., and Porter, A. J. (2003). Selection and characterization of naturally occurring single-domain (IgNAR) antibody fragments from immunized sharks by phage display. Mol.Immunol. 40: 25-33.
Goldman, E. R., Anderson, G. P., Liu, J. L., Delehanty, J. B., Sherwood, L. J., Osborn, L. E., Cummins, L. B., and Hayhurst, A. (2006). Facile generation of heat-stable antiviral and antitoxin single domain antibodies from a semisynthetic llama library. Anal.Chem. 78: 8245-8255.
Griffiths, A. D., Williams, S. C., Hartley, O., Tomlinson, I. M., Waterhouse, P., Crosby, W. L., Kontermann, R. E., Jones, P. T., Low, N. M., Allison, T. J., and et al (1994) Isolation of high affinity human antibodies directly from large synthetic repertoires. EMBO J 13, 3245-3260.
Harmsen, M. M., Ruuls, R. C., Nijman, I. J., Niewold, T. A., Frenken, L. G. J., and de Geus, B. (2000). Llama heavy-chain V regions consist of at least four distinct subfamilies revealing novel sequence features. Mol.Immunol 37: 579-590.
Harrison, J. L., Williams, S. C., Winter, G., and Nissim, A. (1996). Screening of phage antibody libraries. Methods Enzymol. 267: 83-109.
Ho, S. N., Hunt, H. D., Horton, R. M., Pullen, J. K., and Pease, L. R. (1989). Site-directed mutagenesis by overlap extension using the polymerase chain reaction. Gene 77: 51-59.
Holliger, P. and Hudson, P. J. (2005). Engineered antibody fragments and the rise of single domains. Nat.Biotechnol. 23: 1126-1136.
Holt, L. J., Herring, C., Jespers, L. S., Woolven, B. P., and Tomlinson, I. M. (2003). Domain antibodies: proteins for therapy. Trends Biotechnol. 21: 484-490.
Hoogenboom, H. R., Griffiths, A. D., Johnson, K. S., Chiswell, D. J., Hudson, P., and Winter, G. (1991). Multi-subunit proteins on the surface of filamentous phage: methodologies for displaying antibody (Fab) heavy and light chains. Nucleic Acids Res. 19: 4133-4137.
Hoogenboom, H. R. (2002) Overview of antibody phage-display technology and its applications. Methods Mol Biol 178, 1-37
Hoogenboom, H. R. (2005) Selecting and screening recombinant antibody libraries. Nature Biotechnol 23, 1105-1116.
Huie, M. A., Cheung, M. C., Muench, M. O., Becerril, B., Kan, Y. W., and Marks, J. D. (2001). Antibodies to human fetal erythroid cells from a nonimmune phage antibody library. Proc.Natl.Acad.Sci.U.S.A 98: 2682-2687.
Jespers, L., Schon, O., Famm, K., and Winter, G. (2004a). Aggregation-resistant domain antibodies selected on phage by heat denaturation. Nat.Biotechnol. 22: 1161-1165.
Jespers, L., Schon, O., James, L. C., Veprintsev, D., and Winter, G. (2004b). Crystal Structure of HEL4, a Soluble, Refoldable Human V(H) Single Domain with a Germ-line Scaffold. J.Mol.Biol. 337: 893-903.
Kabat, E. A., Wu, T. T., Perry, H. M., Gottesman, K. S. and Foeller, C. (1991). Sequences of Proteins of Immunological Interest. US Department of Health and Human Services, US Public Health Service, Bethesda, Md.
Kirsch, M., Zaman, M., Meier, D., Dubel, S., and Hust, M. (2005). Parameters affecting the display of antibodies on phage. J Immunol Methods 301: 173-185.
Ladenson, R. C., Crimmins, D. L., Landt, Y., and Ladenson, J. H. (2006). Isolation and characterization of a thermally stable recombinant anti-caffeine heavy-chain antibody fragment. Anal.Chem. 78: 4501-4508.
Laune D, Molina F, Ferrieres G, Mani J C, Cohen P, Simon D, Bernardi T, Piechaczyk M, Pau B, Granier C. (1997) Systematic exploration of the antigen binding activity of synthetic peptides isolated from the variable regions of immunoglobulins. J Biol Chem. 272(49):30937-44
Lauwereys, M., Arbabi-Ghahroudi, M., Desmyter, A., Kinne, J., Holzer, W., De Genst, E., Wyns, L., and Muyldermans, S. (1998). Potent enzyme inhibitors derived from dromedary heavy-chain antibodies. EMBO J. 17: 3512-3520.
Liu, J. L., Anderson, G. P., Delehanty, J. B., Baumann, R., Hayhurst, A., and Goldman, E. R. (2007). Selection of cholera toxin specific IgNAR single-domain antibodies from a naive shark library. Mol.Immunol. 44: 1775-1783.
Marks, J. D., Hoogenboom, H. R., Bonnert, T. P., McCafferty, J., Griffiths, A. D., and Winter, G. (1991) By-passing immunization. Human antibodies from V-gene libraries displayed on phage. J Mol Biol 222, 581-597.
Martin, F., Volpari, C., Steinkuhler, C., Dimasi, N., Brunetti, M., Biasiol, G., Altamura, S., Cortese, R., De Francesco, R., and Sollazzo, M. (1997). Affinity selection of a camelized V(H) domain antibody inhibitor of hepatitis C virus NS3 protease. Protein Eng 10: 607-614.
Muyldermans S. Single domain camel antibodies: current status. (2001) J Biotechnol. 74(4):277-302.
Muyldermans, S., Atarhouch, T., Saldanha, J., Barbosa, J. A., and Hamers, R. (1994). Sequence and structure of VH domain from naturally occurring camel heavy chain immunoglobulins lacking light chains. Protein Eng 7: 1129-1135.
Neurath H. (1989) Proteolytic processing and physiological regulation. Trends Biochem Sci. 14(7):268-71.
Nguyen, V. K., Hamers, R., Wyns, L., and Muyldermans, S. (2000). Camel heavy-chain antibodies: diverse germline VHH and specific mechanisms enlarge the antigen-binding repertoire. EMBO J 19: 921-930.
O'Connell, D., Becerril, B., Roy-Burman, A., Daws, M., and Marks, J. D. (2002). Phage versus phagemid libraries for generation of human monoclonal antibodies. J.Mol.Biol. 321: 49-56.
Paz K, Brennan L A, Iacolina M, Doody J, Hadari Y R, Zhu Z (2005)Human single-domain neutralizing intrabodies directed against Etk kinase: a novel approach to impair cellular transformation Mol Cancer Ther. 4(11):1801-9.
Perez, J. M. J., Renisio, J. G., Prompers, J. J., van Platerink, C. J., Cambillau, C., Darbon, H., and Frenken, L. G. J. (2001). Thermal unfolding of a llama antibody fragment: A two-state reversible process. Biochemistry 40: 74-83.
Randen, I., Potter, K. N., Li, Y., Thompson, K. M., Pascual, V., Forre, O. Natvig, J. B., and Capra, J. D. (1993). Complementarity-determining region 2 is implicated in the binding of staphylococcal protein A to human immunoglobulin VHIII variable regions. Eur.J.Immunol. 23: 2682-2686.
Riechmann L, Muyldermans SSingle domain antibodies: comparison of camel VH and camelised human VH domains.J Immunol Methods. 1999 Dec 10;231(1-2):25-38.
Rondot, S., Koch, J., Breitling, F., and Dubel, S. (2001). A helper phage to improve single-chain antibody presentation in phage display. Nat.Biotechnol. 19: 75-78.
Saerens, D., Kinne, J., Bosmans, E., Wernery, U., Muyldermans, S., and Conrath, K. (2004). Single domain antibodies derived from dromedary lymph node and peripheral blood lymphocytes sensing conformational variants of prostate-specific antigen. J.Biol.Chem. 279: 51965-51972.
Sambrook, J., Fritsch, E. F., and Maniatis, T. (1989). “Molecular Cloning: A laboratory Manual, 2nd Ed.”, Cold Spring Harbor Laboratory, Cold Spring Harbor, N.Y.
Shusta, E. V., Raines, R. T., Pluckthun, A., and Wittrup, K. D. (1998). Increasing the secretory capacity of Saccharomyces cerevisiae for production of single-chain antibody fragments. Nat.Biotechnol. 16: 773-777.
Sidhu, S. S., Li, B., Chen, Y., Fellouse, F. A., Eigenbrot, C., and Fuh, G. (2004) Phage-displayed antibody libraries of synthetic heavy chain complementarity determining regions. J Mol. Biol. 338, 299-310.
Soltes, G., Barker, H., Marmai, K., Pun, E., Yuen, A., Wiersma, E. J. (2003) J Immunol Methods 274, 233-244.
Stanfield, R. L., Dooley, H., Flajnik, M. F., and Wilson, I. A. (2004). Crystal structure of a shark single-domain antibody V region in complex with lysozyme. Science 305: 1770-1773.
Stanfield, R. L., Dooley, H., Verdino, P., Flajnik, M. F., and Wilson, I. A. (2007). Maturation of shark single-domain (IgNAR) antibodies: evidence for induced-fit binding. J.Mol.Biol. 367: 358-372.
Streltsov, V. A., Carmichael, J. A., and Nuttall, S. D. (2005). Structure of a shark IgNAR antibody variable domain and modeling of an early-developmental isotype. Protein Sci. 14: 2901-2909.
Tanha, J., Dubuc, G., Hirama, T., Narang, S. A., and MacKenzie, C. R. (2002). Selection by phage display of llama conventional V(H) fragments with heavy chain antibody V(H)H properties. J.Immunol.Methods 263: 97-109.
Tanha, J., Muruganandam, A., and Stanimirovic, D. (2003). Phage display technology for identifying specific antigens on brain endothelial cells. Methods Mol.Med. 89: 435-449.
Tanha, J., Nguyen, T. D., Ng, A., Ryan, S., Ni, F., and MacKenzie, R. (2006). Improving solubility and refolding efficiency of human V(H)s by a novel mutational approach. Protein Eng Des Sel 19: 503-509.
Tanha, J., Xu, P., Chen, Z. G., Ni, F., Kaplan, H., Narang, S. A., and MacKenzie, C. R. (2001). Optimal design features of camelized human single-domain antibody libraries. J.Biol.Chem 276: 24774-24780.
Thomassen, Y. E., Meijer, W., Sierkstra, L., and Verrips, T. (2002). Large-scale production of VHH antibody fragments by Saccharomyces cerevisiae. Enzyme and Microbial Technology 30: 273-278.
To, R., Hirama, T., Arbabi-Ghahroudi, M., MacKenzie, R., Wang, P., Xu, P., Ni, F., and Tanha, J. (2005). Isolation of monomeric human V(H)s by a phage selection. J Biol.Chem 280: 41395-41403.
Transue, T. R., De Genst, E., Ghahroudi, M. A., Wyns, L., and Muyldermans, S. (1998). Camel single-domain antibody inhibits enzyme by mimicking carbohydrate substrate. Proteins 32: 515-522.
van den Beucken T, van Neer N, Sablon E, Desmet J, Celis L, Hoogenboom HR, Hutton S E. Building novel binding ligands to B7.1 and B7.2 based on human antibody single variable light chain domains J Mol Biol. 2001 Jul 13;310(3):591-601
van der Linden, R. H., Frenken, L. G., de Geus, B., Harmsen, M. M., Ruuls, R. C., Stok, W., de Ron, L., Wilson, S., Davis, P., and Verrips, C. T. (1999). Comparison of physical chemical properties of llama VHH antibody fragments and mouse monoclonal antibodies. Biochim.Biophys.Acta 1431: 37-46.
Vaughan, T. J., Williams, A. J., Pritchard, K., Osbourn, J. K., Pope, A. R., Earnshaw, J. C., McCafferty, J., Hodits, R. A., Wilton, J., and Johnson, K. S. (1996). Human antibodies with sub-nanomolar affinities isolated from a large non-immunized phage display library. Nat.Biotechnol. 14: 309-314.
Vieira, J. and Messing, J. (1987). Production of single-stranded plasmid DNA. Methods Enzymol. 153: 3-11.
Vranken, W., Tolkatchev, D., Xu, P., Tanha, J., Chen, Z., Narang, S., and Ni, F. (2002). Solution structure of a llama single-domain antibody with hydrophobic residues typical of the VH/VL interface. Biochemistry 41: 8570-8579.
Vu, K. B., Arbabi-Ghahroudi, M., Wyns, L., and Muyldermans, S. (1997). Comparison of llama VH sequences from conventional and heavy chain antibodies. Mol.Immunol 34: 1121-1131.
Winter, G., Griffiths, A. D., Hawkins, R. E., and Hoogenboom, H. R. (1994). Making antibodies by phage display technology. Annu.Rev Immunol 12: 433-455.
Yau, K. Y., Dubuc, G., Li, S., Hirama, T., MacKenzie, C. R., Jermutus, L., Hall, J. C., and Tanha, J. (2005). Affinity maturation of a V(H)H by mutational hotspot randomization. J Immunol Methods 297: 213-224.
Zhao B, Helms L R, DesJarlais R L, Abdel-Meguid S S, Wetzel R. (1995) A paradigm for drug discovery using a conformation from the crystal structure of a presentation scaffold. Nat Struct Biol. 2(12):1131-7.

APPENDIX
Sequences

HVHP430:
SEQ ID NO 1
(FIG. 2a)
QVQLVESGGGLIKPGGSLRLSCAASGFTFSNYAMSWVRQAPGKGLEWVSA
ISSSGGSTYYADSVKGRFTISRDNSKNTVYLQMNSLRAEDTAVYYCVREE
YRCSGTSCPGAFDIWGQGTMVTVSS
SEQ ID NOs: 2-3
FIG. 3
SEQ ID NOs: 4-11
Table 1
SEQ ID NO 12
FIG. 6A
SEQ ID NOs: 13-14
Example 3, 5^{th} para.
huVHAm302:
SEQ ID NO 15
QVQLVESGGGLIKPGGSLRLSCAASGDTVSDESMTWVRQAPGKGLEWVSA
ISSSGGSTYYADSVKGRFTISRDNSKNTVYLQMNSLRAEDTAVYYCVTDN
RSCQTSLCTSTTRSWGQGTMVTVSS
huVHAm304:
SEQ ID NO 16
VQLVESGGGLIEPGGSLRLSCAASGFSFSDEGMAWVRQAPGKGLEWVSAI
SSSGGSTYYADSVKGRFTISRDNSKNTVYLQMNSLRAEDTAVYYCVRLPK
QCTSPDCETEVSSWGQRTMVTVSS
huVHAm309:
SEQ ID NO 17
QVQLVESGGGLIKPGGSLRLSCAASGVNFSNEGMAWVRQAPGKGLEWVSA
ISSSGGSTYYADSVKGRFTISRDNSKNTVYLQMNSLRAEDTAVYYCVTAQ
RACANSPCPGSITSWGQETMVTVSS
huVHAm315:
SEQ ID NO 18
QVQLVESGGGLIKPGGSLRLSCAASGDMFSSEGMAWVRQAPGKGLEWVSA
ISSSGGSTYYADSVKGRFTISRDNSKNTVYLQMNSLRAEDTAVYYCVAAP
TTCTSHNCAEPFRSWGQETMVTVSS
huVHAm316:
SEQ ID NO 19
QVQLVESGGGLIKPGGSLRLSCAASGDRFTYESMGWVRQAPGKGLEWVSA
ISSSGGSTYYADSVKGRFTISRDNSKNTVYLQMNSLRAEDTAVYYCVALE
TACTRPACAHTPRFWGQGTMVTVSS
huVHAm416:
SEQ ID NO 20
QVQLVESGGGLIKPGGSLRLSCAASGVSFTDDCMAWVRQAPGKGLEWVSA
ISSSGGSTYYADSVKGRFTISRDNSKNTVYLQMNSLRAEDTAVYYCVADH
TQCRQPECESQLCSWGQGTMVTVSS
huVHAm427:
SEQ ID NO 21
QVQLVESGGGLIKPGGSLRLSCAASGVTLSPECMAWVRQAPGKGLEWVSA
ISSSGGSTYYADSVKGRFTISRDNSKNTVYLQMNSLRAEDTAVYYCVSCE
GENAFWGQGTMVTASS
huVHAm428:
SEQ ID NO 22
QVQLVESGGGLIKPGGSLRLSCAASGFSLSDDCMGWVRQAPGKGLEWVSA
ISSSGGSTYYADSVKGRFTISRDNSKNTVYLQMNSLRAEDTAVYYCVTGN
QACKHEPWPDEALLLGPRDNVTVSS
huVHAm431:
SEQ ID NO 23
QVQLVESGGGLIKPGGSLRLSCAASGYTVSSECMGWVRQAPGKGLEWVSA
ISSSGGSTYYADSVKGRFTISRDNSKNTVYLQMNSLRAEDTAVYYCVRDS
KNCHDKDCTRPYCSWGQGTMVTVSS
SEQ ID NOs: 24-90
FIG. 12A
SEQ ID NOs: 91-100
Table 3
SEQ ID NOs: 101-162
Table 4
huVHAm301:
SEQ ID NO 163
QVQLVESGGGLIKPGGSLRLPCAASGFRISHEGMGWVRQAPGKGLEWVSA
ISSSGGSTYYADSVKGRFTISRDNSKNTAYLQMNSLRAEDTAVYYCVAYN
EECTKPSCHTKARSWGQGTMVTVSS
huVHAm303:
SEQ ID NO 164
QVQLVESGGGLIKPGGSLRLSCAASGFRFSYEVMGWVRQAPGKGLEWVSA
ISSSGGSTYYADSVKGRFTISRDNSKNTVYLQMNSLRAEDTAVYYCVTPK
VDCETHPCRERPYFWGQGTMVTVSS
huVHAm305:
SEQ ID NO 165
QVQLVESGGGLIKPGGSLRLSCAASGYRFNNEVMGWVRQAPGKGLEWVSA
ISSSGGSTYYADSVKGRFTISRDNSKNTVYLQMNSLRAEDTAVYYCVTST
PACNQDKCERWRPSWGQGTMVTASS
huVHAm307:
SEQ ID NO 166
QVQLVESGGGLIKPGGSLRLSCAASGFSVSDEDMGWVRQAPGKGLEWVSA
ISSSGGSTYYADSVKGRFTISRDNSKNIVYLQMNSLRAEDTAVYYCVTPL
PKCTNPNCKSPPKYWGQETMVTVSS
huVHAm311:
SEQ ID NO 167
QVQLVESGGGLIKPGGSLRLSCAASGFRVTPECMTWVRQAPGKGLEWVSA
ISSSGGSTYYADSVKGRFTISRDNSKNTVYLQMNSLRAEDTAVYYCVRHE
VECPTEQCPFHCPSWGQGTMVTVSS
huVHAm312:
SEQ ID NO 168
QVQLVESGGGLIKPGGSLRLSCAASGVMGWVRQAPGKGLEWVSAISSSGG
STYYADSVKGRFTISRDNSKNTVYLQMNSLRAEDTAVYYCVAPETQCSEG
RCLGTASSWGQGTMVTVSS
huVHAm313:
SEQ ID NO 169
QVQLVESGGGLIKPGGSLRLSCAASGFRFIDEDMGWVRQAPGKGLEWVSA
ISSSGGSTYYADSVKGRFTISRDNSKNTVYLQMNSLRAEDTAVYYCVAGA
KGQWSPSLQAQAGQ
huVHAm317:
SEQ ID NO 170
QVQLVESGGGLIKPGGSLRLSCAASGYMISDEIMAWVRQAPGKGLEWVSA
ISSSGGSTYYADSVKGRFTISRDNSKNTVYLQMNSLRAEDTAVYYCVAPN
RAKGQWSTVSS
huVHAm320:
SEQ ID NO 171
QVQLVESGGGLIKPGGSLRLSCAASGYSVSDESMGWVRQAPGKGLEWVSA
ISSSGGSTYYADSVKGRFTISRDNSKNTVYLQMNSLRAEDTAVYYCVTTD
PLGAKGQWSPSSSGQAGQ
huVHAm406:
SEQ ID NO 172
QVQLVESGGGLIKPGGSLRLSCAASGFSFTPECMGWVRQAPGKGLEWVSA
ISSSGGSTYYADSVKGRFTISRDNSKNTVYLQMNSLRAEDTAVYYCVGHK
NNCPGQGTMVTVSS
huVHAm412:
SEQ ID NO 173
QVQLVESGGGLIKPGGSLRLSCAASGDMLSAECMGWVRQAPGKGLEWVSA
ISSSGGSTYYADSVKGRFTISRDNSKNTVYLQMNSLRAEDTAVYYCVAKP
YHCAVQGTMVTVSS
huVHAm420:
SEQ ID NO 174
QVQLVESGGGLIKPGGSLRLSCAASGDRFSYEDMAWVPQAPGKGLEWVSA
ISSSGGSTYYADSVKGRFTISRDNSKNTVYLQMNSLRAEDTAVYYCVATE
ESCPEGNCPPPRRSWGQETMVTVSS
huVHAm424:
SEQ ID NO 175
QVQLVESGGGLIKPGGSLRLSCAASGDRVISECMGWVSAISSSGGSTYYA
DSVKGRFTISRDNSKNTVYLQMNSLRAEDTAVYYCVALPPEVCEADVPDR
GDLLGPRTMVTVSS
huVHAm430:
SEQ ID NO 176
QVQLVESGGGLIKPGGSLRLSCAASGDRVSPEDMAWVRQAPGKGLEWVSA
ISSSGGSTYYADSVKGRFTISRDNSKNTVYLQMNSLRAEDTAVYYCVTSG
VPSGSFWGQETMVTVSS
SEQ ID NOs: 177-178
Figure legend of fIG. 2A
SEQ ID NOs: 179-181
Figure legend of FIG. 6
SEQ ID NOs: 182-184
FIG. 14

1. A non-aggregating V_{H} domain and libraries thereof, the V_{H} domain comprising at least one disulfide linkage-forming cysteine in at least one complementarity-determining region (CDR) and comprising an acidic isoelectric point (pI).

2. The non-aggregating V_{H} domain and libraries thereof of claim 1, wherein the V_{H} domains are soluble, capable of reversible thermal unfolding, and/or capable of binding to protein A.

3-4. (canceled)

5. The non-aggregating V_{H} domain and libraries thereof of claim 1, wherein the V_{H} domains comprises non-canonical disulfide linkages within one CDR or between CDRs.

6. (canceled)

7. The non-aggregating V_{H} domain and libraries thereof of claim 1, wherein the V_{H} domains comprise an acidic amino acid residue at position 32 of CDR1.

8. The non-aggregating V_{H} domain and libraries thereof of claim 1, wherein the V_{H} domains comprise an isoelectric point of below 6.

9. The non-aggregating V_{H} domain and libraries thereof of claim 1, wherein the V_{H} domains comprise a sequence selected from any one of SEQ ID NOs:24-90, SEQ ID NOs:101-131, SEQ ID NOs: 132-162, and combinations thereof.

10. The non-aggregating V_{H} domain and libraries thereof of claim 1, wherein the V_{H} domains comprise human framework sequences and at least one CDR from a different species.

11. The non-aggregating V_{H} domain and libraries thereof of claim 10, wherein the V_{H} domains comprise human framework sequences, human CDR1/HI, human CDR2/H2, and camelid CDR3/H3.

12. (canceled)

13. The non-aggregating V_{H} domain and libraries thereof of claim 1, wherein the V_{H} domains are enzyme inhibitors.

14. The non-aggregating V_{H} domain and libraries thereof of claim 1, wherein the V_{H} domains are based on the human germline sequences 1-f V_{H} segment, 1-24 V_{H} segment and 3-43 V_{H} segment.

15. The non-aggregating V_{H} domain and libraries thereof of claim 1, wherein the V_{H} domains are based on human germline sequences with acidic pI, camelid V_{H} cDNAs, camelid germline V_{H} segments with acidic pIs, camelid V_{H}H cDNAs, or camelid germline V_{H}H segments with acidic pIs.

16. The non-aggregating V_{H} domain and libraries thereof of claim 1, wherein the V_{H} domain is selected from the group consisting of huVHAm302, huVHAm309, huVHAm316, huVHAm303, huVHAm304, huVHAm305, huVHAm307, huVHAm311, huVHAm315, huVHAm301, huVHAm312, huVHAm320, huVHAm317, huVHAm313, huVHAm431, huVHAm427, huVHAm416, huVHAm424, huVHAm428, huVHAm430, huVHAm406, huVHAm412, and huVHAm420.

17-18. (canceled)

19. A method of increasing the power or efficiency of selection of non-aggregating V_{H} domains, comprising:
a) providing a phagemid-based V_{H} domain phage-display library, wherein the library is produced by multivalent display of V_{H} domains on the surface of phage; and
b) panning, using the phage- V_{H} domain library and a target,
wherein the method comprises a step of selection of non-aggregating phage-V_{H} domains.

20. (canceled)

21. The method of claim 19, wherein the selection step is a step of sequencing individual clones to identify the V_{H} with acidic pIs, the selection step occurring following the step of panning (step b)).

22-23. (canceled)

24. The method of claim 13, further comprising a step of isolating specific V_{H} domains from the phagemid-based V_{H} domain phage-display library.

25. A method of increasing the power or efficiency of selection of non-aggregating V_{H} domains, comprising:
a) providing a phage vector-based V_{H} domain phage-display library, wherein the library is produced based on a V_{H} domain scaffold having an acidic pI;
b) panning, using the phage-V_{H} domain library and a target; and
c) sequencing individual clones to identify V_{H} domains having an acidic pI.

26. The method of claim 25, wherein the V_{H} domain scaffolds are based on human germline sequences with acidic pI, camelid V_{H} cDNAs, camelid germline V_{H} segments with acidic pIs, camelid V_{H}H cDNAs, or camelid germline V_{H}H segments with acidic pIs.

27. The method of claim 25, further comprising a step of isolating specific V_{H} domains from the phage vector-based V_{H} domain phage-display library.

28. A nucleic acid encoding a V_{H} domain of claim 1.

29. A vector comprising the nucleic acid of claim 28.

30. (canceled)

31. A pharmaceutical composition comprising an effective amount of one or more than one V_{H} domain of claim 1 for binding to an antigen, and a pharmaceutically-acceptable excipient.

32. (canceled)

33. A method of treating a patient comprising administering a pharmaceutical composition comprising one or more than one V_{H} domain of claim 1 to a patient in need of treatment.

34. A kit comprising one or more than one V_{H} domain of claim 1 and one or more reagents, for detection and determination of binding of the one or more than one V_{H} domain to a particular antigen in a biological sample.

35. (canceled)

36. The V_{H} domain or library thereof of claim 1, wherein a) the V_{H} domain is based on HVHP430 (SEQ ID NO:1); b) the Cys at positions 99 and 100d of CDR3 are maintained; c) the remaining 14 amino acid residues of CDR3 are randomized; d) amino acid residue 94 is randomized; and e) the 8 amino acid residues of CDR1/H1 are randomized.

37. The V_{H} domain or library thereof of claim 1, wherein a) the V_{H} domain is based on HVHP430 (SEQ ID NO:1); b) the amino acid residues at 93-102 (93/94-CDR3) positions are derived from llama V_{H}Hs; c) the 8 amino acid residues of CDR1/H1 are randomized.

38. The V_{H} domain or library thereof of claim 1, wherein a) the V_{H} domain is based on HVHP430 (SEQ ID NO:1); b) the CDR3 comprises a sequence selected from SEQ ID NOs:24-90 and SEQ ID NOs:33-63; c) the 8 amino acid residues of CDR1/H1 are randomized.

39. The V_{H} domain or library thereof of claim 1 coupled to a cargo molecule, or labelled with a detectable label of marker.
